(19) United States
(12) Patent Application Publication
(10) Pub. No.: US 2025/0352487 A1
(43) Pub. Date: Nov. 20, 2025

(54) Lipid Nanoparticle Formulations for Central Nervous System Delivery

(71) Applicant: Carnegie Mellon University, Pittsburgh, PA (US)

(72) Inventors: Kathryn Whitehead, Pittsburgh, PA (US); Saigopalakrishna Saileelaprasad Yerneni, Pittsburgh, PA (US)

(21) Appl. No.: 18/862,781

(22) PCT Filed: May 4, 2023
(86) PCT No.: PCT/US23/20980

§ 371 Date: Nov. 4, 2024

Related U.S. Application Data
(60) Provisional application No. 63/338,216, filed on May 4, 2022.

(51) Int. Cl. A61K 9/51; A61K 9/00; A61K 47/54; A61K 47/69; A61K 48/00; C12N 15/88

(52) U.S. Cl. CPC A61K 9/5123; A61K 9/0019; A61K 9/0085; A61K 9/5146; A61K 47/543; A61K 47/6929; A61K 48/0075; C12N 15/88

(57) ABSTRACT

Provided herein are compositions and methods of delivering nucleic acids, such as therapeutic mRNAs and DNA, to the central nervous system by delivery, e.g., intrathecally or intracerebrally, of nucleic acid containing lipid nanoparticles using sphingolipids as helper lipids.

### CROSS REFERENCE TO RELATED APPLICATIONS

This application claims the benefit of U.S. Provisional Patent Application No. 63/338,216 filed May 4, 2022, the disclosure of which is incorporated herein by reference in its entirety.

Lipid nanoparticles are efficient carriers of cargo, such as a nucleic acid cargo, for delivery into cells for gene delivery, mRNA delivery, antisense, RNA interference, among other uses. Lipid nanoparticles typically comprise helper lipids, cholesterol, ionizable lipids (e.g., lipidoids), lipid-polymer conjugates and nucleic acid cargo. Lipid nanoparticles are typically administered in an intravenous, intramuscular or subcutaneous injection. Exemplary LNP compositions and/or compositions, e.g., lipidoids, useful in producing LNPs are described in U.S. Pat. Nos. 10,844,028, 10,189,802, 9,872,911, 9,556,110, 9,439,968, 9,227,917, 8,969,353, and 8,450,298, as well as in U.S. Patent Application Publication Nos. 2017/0204075, 2019/0177289, 2017/0152213, 2016/0114042, 2015/0203439, 2014/0322309, 2014/0161830, 2011/0293703, and 2010/0331234, each of which is incorporated herein by reference for its technical disclosure relating to compounds and compositions useful in delivery of nucleic acid cargos, and to the extent it is consistent with the present disclosure. Additional examples of lipid nanoparticles are described in U.S. Pat. Nos. 9,404,127, 9,364,435, and 8,058,069, each of which incorporated herein by reference for its technical disclosure relating to compounds and compositions useful in delivery of nucleic acid cargoes, and to the extent it is consistent with the present disclosure (see, also, e.g., Sabnis S, et al., A Novel Amino Lipid Series for mRNA Delivery: Improved Endosomal Escape and Sustained Pharmacology and Safety in Non-human Primates. Mol Ther. 2018; 26 (6):1509-1519 and Yonezawa S, et al., Recent advances in siRNA delivery mediated by lipid-based nanoparticles. Adv Drug Deliv Rev. 2020; 154-155:64-78). Examples of lipid nanoparticles, lipidoids, and methods of making lipid nanoparticles and lipidoids, as described herein, are described in Whitehead K A, et al., Degradable lipid nanoparticles with predictable in vivo siRNA delivery activity. Nat Commun. 2014 Jun. 27; 5:4277. doi: 10.1038/ncomms5277. PMID: 24969323; PMCID: PMC4111939.

Despite successes in parenteral delivery of nucleic acids via LNPs, there are significant obstacles to delivery of nucleic acids to cells of the central nervous system (CNS). Vehicles for effective delivery of nucleic acids to the cells of the central nervous system are needed.

### SUMMARY

According to a first embodiment or aspect of the invention, a method of delivery of a therapeutic agent to tissue of the central nervous system (CNS) of a patient is provided. The method comprises administering to tissue of the patient's CNS a composition comprising a lipid-containing particle, comprising a therapeutic agent, the lipid-containing particle further comprising: a sphingolipid helper lipid; cholesterol or a derivative thereof; a PEG-based compound, such as a PEG-containing polymer or a PEGylated fatty acid-containing compound; and an ionizable lipidoid.

According to a further embodiment or aspect of the invention, a lipid-containing particle is provided. The lipid-containing particle comprises a therapeutic agent, the lipid-containing particle further comprising: a sphingolipid helper lipid; cholesterol or a derivative thereof; a PEG-based compound, such as a PEG-containing polymer or a PEGylated fatty acid-containing compound; and an ionizable lipidoid.

The following numbered clauses outline various aspects or embodiments of the present invention.

Clause 1. A method of delivery of a therapeutic agent to tissue of the central nervous system (CNS) of a patient, comprising administering to tissue of the patient's CNS a composition comprising a lipid-containing particle, comprising a therapeutic agent, the lipid-containing particle further comprising: a sphingolipid helper lipid; cholesterol or a derivative thereof; a PEG-based compound, such as a PEG-containing polymer or a PEGylated fatty acid-containing compound; and an ionizable lipidoid.

Clause 2. The method of clause 1, wherein the lipid-containing particle is a lipid nanoparticle.

Clause 3. The method of clause 1 or 2, wherein the lipid-containing particle is administered intrathecally, intracerebrally, or to a patient's brainstem.

Clause 4. The method any one of clauses 1-3, wherein the sphingolipid is a ceramide, a sphingomyelin, a ceramide phosphoethanolamine, a sphingosyl-phosphorylcholine, a sphingosine, a glycosphingolipid, or any combination of two or more of the preceding.

Clause 5. The method of clause 4, wherein the sphingolipid is a ceramide, a glucosyl ceramide, a galactosyl ceramide, or a lactosyl ceramide.

Clause 6. The method of any one of clauses 1-5, wherein the sphingolipid is a CNS sphingolipid, a brain sphingolipid, ceramide, lactosyl ceramide, galactosyl ceramide, glucosyl ceramide or a combination of any two or more of the preceding.

Clause 7. The method of any one of clauses 1-5, wherein the sphingolipid is ceramide, lactosyl ceramide, galactosyl ceramide, or a combination of any two or more of the preceding.

Clause 8. The method of any one of clauses 1-7, wherein the lipidoid is one or more of 306_{Oi10}; 306O₁₀; 503Oᵢ₁₀; 402O_{6,10}; 500X₁; 500Oᵢ₁₀; 306O₁₁; 306Oᵢ₁₀; 306O₁₂; 200X₆; 516Oᵢ₁₀; 500O_{1,1,8}; 514X₆; 306O₁₄; 501X₁; 205O₁₆; 500O₁₃; 113Oᵢ₁₀; 306O₁₆; 306O₁₃; 205O₁₈; 509X₇; 501Oᵢ₁₀; 503Oᵢ₁₀; 500O₁₄; 113Oᵢ₁₀; 509X₁; 509X₃; 501X₂; 402O_{6,10}; 516O_{4,8}; 402X₈; 501O_{1,1,8}; or 509O_{1,1,8}.

Clause 9. The method of any one of clauses 1-7, wherein the lipidoid is one or more of 306_{Oi10}, 306O₁₀, 503Oᵢ₁₀, and 402O_{6,10}.

Clause 10. The method of clause 1, wherein the ionizable lipidoid is 306_{Oi10}.

Clause 11. The method of any one of clauses 1-10, wherein the therapeutic agent is anionic or polyanionic.

Clause 12. The method of any one of clauses 1-10, wherein the therapeutic agent is a nucleic acid.

Clause 13. The method of clause 12, wherein the nucleic acid comprises an RNA.

Clause 14. The method of clause 13, wherein the RNA comprises an mRNA.

Clause 15. The method of clause 13, wherein the RNA comprises an RNAi reagent, a dsRNA, an siRNA, an shRNA, a miRNA, an antisense RNA, a guide RNA (gRNA), a long non-coding RNAs (lncRNA), a base editing gRNA (beRNA), a prime editing gRNA (pegRNA), or a transfer RNA (tRNA).

Clause 16. The method of clause 13, wherein the RNA comprises gRNA, beRNA, or pegRNA and an mRNA encoding Cas9, or a Cas9 fusion protein for base editing or prime editing.

Clause 17. The method of clause 12, wherein the nucleic acid encodes a heme oxygenase-1 (HO1), brain-derived neurotrophic factor (BDNF), or tumor necrosis factor-related apoptosis-inducing ligand (TRAIL) protein.

Clause 18. The method of clause 12, wherein the nucleic acid is DNA.

Clause 19. The method of any one of clauses 1-17, wherein the lipid-containing particle is a lipid nanoparticle, comprising, by mol % of the sphingolipid helper lipid, the cholesterol or a derivative thereof, the PEG-based compound, and the lipidoid: from 5 to 95 mol % of the sphingolipid helper lipid; from 5 to 75 mol % of the cholesterol or a derivative thereof; from 0.1 to 50 mol % of the PEG-based compound; and from 5 to 90 mol % of the ionizable lipidoid.

Clause 20. The method of any one of clauses 1-19, wherein the cholesterol or a derivative thereof is cholesterol.

Clause 21. The method of any one of clauses 1-20, wherein the PEG-based compound is one or more of: PEG-ceramide, PEG-DMG, PEG-PE, poloxamer, or DSPE carboxy PEG. For instance, in certain embodiments, the PEG-based material is C14 PEG2000 DMG, C15 PEG2000 DMG, C16 PEG2000 DMG, C18 PEG2000 DMG, C14 PEG 2000 ceramide, C15 PEG2000 ceramide, C16 PEG2000 ceramide, C18 PEG2000 ceramide, C14 PEG2000 PE, C15 PEG2000 PE, C16 PEG2000 PE, C18 PEG2000 PE, C14 PEG350 PE, C14 PEG5000 PE, poloxamer F-127, poloxamer F-68, poloxamer L-64, and DSPE carboxy PEG.

Clause 22. The method of clause 21, wherein the PEG-based compound comprises a PEGylated fatty acid, such as a PEGylated C10-C20 fatty acid-containing compound, such as C14-PEG2000-PE.

Clause 23. The method of any one of clauses 1-22, wherein the nucleic acid encodes a heme oxygenase-1 (HO1) and/or brain-derived neurotrophic factor (BDNF) for treatment of ischemia or ischemia/reperfusion injury, for example ischemic stroke, in the patient.

Clause 24. The method of any one of clauses 1-22, wherein the nucleic acid encodes a tumor necrosis factor-related apoptosis-inducing ligand (TRAIL) protein, for treatment of a cancer, for example glioblastoma, in the patient.

Clause 25. A lipid-containing particle, comprising a therapeutic agent, the lipid-containing particle further comprising: a sphingolipid helper lipid; cholesterol or a derivative thereof; a PEG-based compound, such as a PEG-containing polymer or a PEGylated fatty acid-containing compound; and an ionizable lipidoid.

Clause 26. The lipid-containing particle of clause 25, wherein the lipid-containing particle is a lipid nanoparticle.

Clause 27. The lipid-containing particle of clause 25 or 26, wherein the sphingolipid is a ceramide, a sphingomyelin, a ceramide phosphoethanolamine, a sphingosyl-phosphorylcholine, a sphingosine, a glycosphingolipid, or any combination of two or more of the preceding.

Clause 28. The lipid-containing particle of clause 27, wherein the sphingolipid is a ceramide, a glucosyl ceramide, a galactosyl ceramide, or a lactosyl ceramide.

Clause 29. The lipid-containing particle of any one of clauses 25-28, wherein the sphingolipid is a CNS sphingolipid, a brain sphingolipid, ceramide, lactosyl ceramide, galactosyl ceramide, glucosyl ceramide or a combination of any two or more of the preceding.

Clause 30. The lipid-containing particle of any one of clauses 25-28, wherein the sphingolipid is ceramide, lactosyl ceramide, galactosyl ceramide, or a combination of any two or more of the preceding.

Clause 31. The lipid-containing particle of any one of clauses 25-30, wherein the lipidoid is one or more of 306_{Oi10}; 306O₁₀; 503Oᵢ₁₀; 402O_{6,10}; 500X₁; 500Oᵢ₁₀; 306O₁₁; 306Oᵢ₁₀; 306O₁₂; 200X₆; 516Oᵢ₁₀; 500O_{1,1,8}; 514X₆; 306O₁₄; 501X₁; 205O₁₆; 500O₁₃; 113Oᵢ₁₀; 306O₁₆; 306O₁₃; 205O₁₈; 509X₇; 501Oᵢ₁₀; 503Oᵢ₁₀; 500O₁₄; 113Oᵢ₁₀; 509X₁; 509X₃; 501X₂; 402O_{6,10}; 516O_{4,8}; 402X₈; 501O_{1,1,8}; or 509O_{1,1,8}.

Clause 32. The lipid-containing particle of any one of clauses 25-30, wherein the lipidoid is one or more of 306_{Oi10}, 306O₁₀, 503Oᵢ₁₀, and 402O_{6,10}.

Clause 33. The lipid-containing particle of clause 25, wherein the ionizable lipidoid is 306_{Oi10}.

Clause 34. The lipid-containing particle of any one of clauses 25-33, wherein the therapeutic agent is anionic or polyanionic.

Clause 35. The lipid-containing particle of any one of clauses 25-33, wherein the therapeutic agent is a nucleic acid.

Clause 36. The lipid-containing particle of clause 35, wherein the nucleic acid comprises an RNA.

Clause 37. The lipid-containing particle of clause 36, wherein the RNA comprises an mRNA.

Clause 38. The lipid-containing particle of clause 36, wherein the RNA comprises an RNAi reagent, a dsRNA, an siRNA, an shRNA, a miRNA, an antisense RNA, a guide RNA (gRNA), a long non-coding RNAs (lncRNA), a base editing gRNA (beRNA), a prime editing gRNA (pegRNA), or a transfer RNA (tRNA).

Clause 39. The lipid-containing particle of clause 36, wherein the RNA comprises gRNA, beRNA, or pegRNA and an mRNA encoding Cas9, or a Cas9 fusion protein for base editing or prime editing.

Clause 40. The lipid-containing particle of clause 35, wherein the nucleic acid encodes a heme oxygenase-1 (HO1), brain-derived neurotrophic factor (BDNF), or tumor necrosis factor-related apoptosis-inducing ligand (TRAIL) protein.

Clause 41. The lipid-containing particle of clause 35, wherein the nucleic acid is DNA.

Clause 42. The lipid-containing particle of any one of clauses 25-41, wherein the lipid-containing particle is a lipid nanoparticle, comprising, by mol % of the sphingolipid helper lipid, the cholesterol or a derivative thereof, the PEG-based compound, and the lipidoid: from 5 to 95 mol % of the sphingolipid helper lipid; from 5 to 75 mol % of the cholesterol or a derivative thereof; from 0.1 to 50 mol % of the PEG-based compound; and from 5 to 90 mol % of the ionizable lipidoid.

Clause 43. The lipid-containing particle of any one of clauses 25-42, wherein the cholesterol or a derivative thereof is cholesterol.

Clause 44. The lipid-containing particle of any one of clauses 25-43, wherein the PEG-based compound is one or more of: PEG-ceramide, PEG-DMG, PEG-PE, poloxamer, or DSPE carboxy PEG. For instance, in certain embodiments, the PEG-based material is C14 PEG2000 DMG, C15 PEG2000 DMG, C16 PEG2000 DMG, C18 PEG2000 DMG, C14 PEG 2000 ceramide, C15 PEG2000 ceramide, C16 PEG2000 ceramide, C18 PEG2000 ceramide, C14 PEG2000 PE, C15 PEG2000 PE, C16 PEG2000 PE, C18 PEG2000 PE, C14 PEG350 PE, C14 PEG5000 PE, poloxamer F-127, poloxamer F-68, poloxamer L-64, and DSPE carboxy PEG.

Clause 45. The lipid-containing particle of clause 44, wherein the PEG-based compound comprises a PEGylated fatty acid, such as a PEGylated C10-C20 fatty acid-containing compound, such as C14-PEG2000-PE.

Clause 46. The lipid-containing particle of any one of clauses 25-45, wherein the nucleic acid encodes a heme oxygenase-1 (HO1) and/or brain-derived neurotrophic factor (BDNF) for treatment of ischemia or ischemia/reperfusion injury, for example ischemic stroke, in the patient.

Clause 47. The lipid-containing particle of any one of clauses 25-45, wherein the nucleic acid encodes a tumor necrosis factor-related apoptosis-inducing ligand (TRAIL) protein, for treatment of a cancer, for example glioblastoma, in the patient.

Clause 48. The lipid-containing particle of any one of clauses 25-45, for treatment of neuroinflammation, a neurodegenerative disease, or a monogenic neurological disorder.

Clause 49. A method of treating a patient having ischemia, stroke, ischemia/reperfusion injury, neuroinflammation, a neurodegenerative disease, a monogenic neurological disorder, or a cancer, comprising administering to the patient an effective amount of the lipid nanoparticle as claimed in any one of clauses 25-45, thereby treating the ischemia, stroke, ischemia/reperfusion injury, neuroinflammation, neurodegenerative disease, monogenic neurological disorder, or a cancer in the patient.

### BRIEF DESCRIPTION OF THE DRAWINGS

FIGS. 1A and 1B. FIG. 1A provides a general reaction scheme between an amine, e.g., the numbered compounds shown in FIGS. 2A-2C, and an acrylate tail, e.g., the compounds designated O_, as shown in FIGS. 3A-3C to form a lipidoid, which is referenced by the reacted amine and the reacted acrylate tail (###O_), e.g., 306O₁₀, as shown in FIG. 1B. The compounds may be prepared by the addition of a primary or secondary amine to an acrylate via a Michael addition reaction.

FIGS. 2A-2C provide exemplary amines for use in preparing lipidoids as described herein.

FIGS. 3A-3C provide exemplary acrylates for use in preparing lipidoids as described herein.

FIGS. 4A-4D provide graphs showing the total integrated green GFP fluorescence intensity for HCM3 cells (FIG. 4A), SIM-A9 cells (FIG. 4B), U87MG cells (FIG. 4C) and GL261 cells (FIG. 4D) treated with LNPs formulated with various helper lipids.

FIGS. 5A-5D provide graphs showing the cell confluence (%) for HCM3 cells (FIG. 4A), SIM-A9 cells (FIG. 4B), U87MG cells (FIG. 4C), and GL261 cells (FIG. 4D) treated with LNPs formulated with various helper lipids.

FIGS. 6A-6B show the kinetics of mLuc expression, where FIG. 6A is a graph showing the quantification of luciferase expression in different organs over a period of two days, where points indicate the mean±standard error of the mean (SEM) (n=3 mice) and FIG. 6B includes IVIS images showing the expression of luciferase in the mouse brain, spinal cord, spleen, liver, pancreas, heart, lungs, and liver at different time points after injection with DOPE helper lipid formulated LNP.

FIGS. 7A-7B show the spatial distribution of intrathecally injected LNPs, where FIG. 7A is a graph showing the quantification of luciferase expression in regions of the brain and spinal cord (n=3 mice) in in C57BL/6 mice that were intrathecally administered DOPE helper lipid LNPs 306_{Oi10} LNPs and FIG. 7B includes IVIS images showing the expression of luciferase in the different regions of the brain after injection with DOPE helper lipid formulated LNP.

FIGS. 8A-8C show the delivery of multiple mRNAs using the intrathecal route, where FIG. 8A includes IVIS images showing the expression of luciferase and mCherry in different mouse organs, FIG. 8B is a graph showing the quantification of luciferase expression in different organs, and FIG. 8C is a graph showing the quantification of mcherry expression. FIGS. 8C and 8B include the mean±SEM (n=3 mice).

FIGS. 9A-9B show that LNPs successfully transfected luciferase mRNA to a mouse brain after a single intrathecal injection, where FIG. 9A provides bioluminescence imaging of the brains and spinal cords from sacrificed mice after intrathecal injection of luciferase mRNA-LNPs at 3 hours and FIG. 9B shows the semiquantitative luciferase activity evaluation of the brain and main organs after intrathecal injection, where percentages indicate mean luminescent flux±SEM (n=3 mice/group).

FIGS. 10A-10I show that LNPs successfully transfected mouse brains with mCre recombinase mRNA after a single intrathecal injection. FIG. 10A provides IVIS images of the biodistribution of mCre-LNPs in mice with the spectrum gradient bar corresponding to the fluorescence intensity and a bar graph showing the relative tdTomato photon flux in the brain after LNP administration. Differences between control and treated groups were determined using one-way ANOVA. *p<0.05. Error bars represent SEM. FIGS. 10B-10I are bar graphs showing the percentages of total cells (FIG. 10B), astrocytes (GFAP+; FIG. 10C), neurons (CD90.2+NeuN+; FIG. 10D), oligodendrocytes (04+; FIG. 10E), tD Tomato Intensity (FIG. 10F), neural stem cells (CD133+; FIG. 10G), lymphocytes (CD45+, CD3+; FIG. 10H), and microglia (CD11b+; FIG. 10I) populations. Data were analyzed by one-way ANOVA followed by Turkey post hoc. * indicates p<0.05.

FIGS. 11A-11L are graphs showing the total expression of Cas9 protein in microglia cells (FIG. 11A), neurons (FIG. 11B), astrocytes (FIG. 11C), oligodendrocytes (FIG. 11D), neutral stem cells (FIG. 11E), and lymphocytes (FIG. 11F) at 24 hours after intrathecal injection and the total expression of CD81 in microglia cells (FIG. 11G), neurons (FIG. 11H), astrocytes (FIG. 11I), oligodendrocytes (FIG. 11J), neutral stem cells (FIG. 11K), and lymphocytes (FIG. 11L) 1 week after intrathecal injection. Bars indicate mean±SEM (n=3 mice).

FIGS. 12A-12C show the delivery of multiple mRNAs using the intrathecal route, where FIG. 12A provides IVIS images showing expression of luciferase and mCherry in different mouse organs. FIG. 12B is a graph showing the quantification of luciferase expression in different organs, and FIG. 12C is a graph showing the quantification of mCherry expression. FIGS. 12C and 12B include the mean±SEM (n=3 mice).

FIGS. 13A-13J show that LNPs formulated with sphingolipids successfully transfected mouse brains with mCre recombinase mRNA after a single intrathecal injection. FIG. 13A provides IVIS images of the biodistribution of mCre-LNPs in mice with the spectrum gradient bar corresponding to the fluorescence intensity and a bar graph showing the relative tdTomato photon flux in the brain after LNP administration.

FIG. 13B is a graph showing the quantification of tdTomato fluorescence shown of FIG. 14A. FIGS. 13C-13J are bar graphs showing the percentages of all cells (FIG. 13C), astrocytes (GFAP+; FIG. 13D), neurons (CD90.2+NeuN+; FIG. 13E), oligodendrocytes (O4+; FIG. 13F), tD Tomato Intensity (FIG. 13G), neural stem cells (CD133+; FIG. 13H), lymphocytes (CD45+, CD3+; FIG. 13H), and microglia (CD11b+; FIG. 13J) populations. Data were analyzed by one-way ANOVA followed by Turkey post hoc. * indicates p<0.05.

FIG. 14 includes photomicrographs of immunostained Ai9 mouse brains from mice that underwent an intrathecal injection of ceramide-LNPs containing mCre. Scale bar=10 um.

FIGS. 15A-15L are graphs showing the total expression of Cas9 protein in different brain cells at 24 hours after intrathecal injection (FIGS. 15A-15F) and CD81 expression levels in different brain cells 1 week after LNP administration (FIGS. 15G-15L). Bars indicate mean±SEM (n=3 mice).

FIGS. 16A-16H show results from gene sequencing that confirm robust Cas9-mediated CD81 gene editing in mouse brains. FIG. 16A shows the amplified CD81 gene in the genomic DNA from mice brain (n=3 per treatment group). FIGS. 16B-16D are combined frequency of insertions/deletions/substitutions in the CD81 gene locus in mice brains. FIGS. 16D-16H show the frequency of indels along the CD81 gene locus as indicated by illumine gene sequencing.

### DETAILED DESCRIPTION

Other than in the operating examples, or where otherwise indicated, the use of numerical values in the various ranges specified in this application are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. Ranges between two numbers, including those two numbers may be alternatively stated as ranging from a first number to a second number, such as, for example, “n ranges from a to b, inclusive” may be alternatively stated as “n is from a to b”. The terms “greater than” or “less than” may be used to exclude stated values, as in “n ranges from greater than a to b”, which excludes a, but includes b (that is, a<n≤b).

As used herein, “a” and “an” refer to one or more.

The term “comprising” is open-ended and may be synonymous with “including”, “containing”, or “characterized by”. The term “consisting essentially of” limits the scope of a claim to the specified materials or steps and those that do not materially affect the basic and novel characteristic(s) of the claimed invention. The term “consisting of” excludes any element, step, or ingredient not specified in the claim. As used herein, embodiments “comprising” one or more stated elements or steps also include, but are not limited to embodiments “consisting essentially of” and “consisting of” those stated elements or steps. For definitions provided herein, those definitions refer to word forms, cognates and grammatical variants of those words or phrases.

As used herein, the terms “patient” or “subject” refer to members of the animal kingdom including but not limited to human beings and “mammal” refers to all mammals, including, but not limited to human beings.

“Treatment” in the context of a disease or disorder, a marker for a disease or a disorder, or a symptom of a disease or disorder, can refer to a clinically-relevant and/or a statistically significant decrease or increase in an ascertained value for a clinically-relevant marker from outside a normal range towards, or to, a normal range. The decrease or increase can be, for example, at least 10%, at least 20%, at least 30%, at least 40%, or more, to a level accepted as either a therapeutic goal, or a level within the range of normal for an individual without such disease or disorder, or, in the case of a lowering of a value, to below the level of detection of an assay. The decrease or increase can be to a level accepted as within the range of normal for an individual without such disease or disorder, which can also be referred to as a normalization of a level. The reduction or increase can be the normalization of the level of a sign or symptom of a disease or disorder, that is, a reduction in the difference between the subject level of a sign of the disease or disorder and the normal level of the sign for the disease or disorder (e.g., to the upper level of normal when the value for the subject must be decreased to reach a normal value, and to the lower level of normal when the value for the subject must be increased to reach a normal level).

The compositions described herein may include as an active agent, a nucleic acid reagent, such as, without limitation, a DNA, an RNA (e.g., an mRNA), an antisense reagent, or an RNAi (RNA interference) reagent.

As used herein, the terms “cell” and “cells” refer to any types of cells from any animal, such as, without limitation, rat, mouse, monkey, and human. For example and without limitation, cells can be progenitor cells, e.g., pluripotent cells, including stem cells, induced pluripotent stem cells, multipotent cells, or differentiated cells, such as endothelial cells and smooth muscle cells. “Cells” may be in vivo, e.g., as part of a tissue or organ, or in vitro, such as a population of cells, such as, for example, a population of cells enriched for a specific cell type, such as, without limitation, a progenitor cell or a stem cell.

A composition is “biocompatible” in that the composition and, where applicable, elements thereof, or degradation products thereof, are substantially non-toxic to cells or organisms within acceptable tolerances, including substantially non-carcinogenic and substantially non-immunogenic, and are cleared or otherwise degraded in a biological system, such as an organism (patient) without substantial toxic effect. Non-limiting examples of degradation mechanisms within a biological system include chemical reactions, hydrolysis reactions, and enzymatic cleavage.

“Therapeutically effective amount,” as used herein, can include the amount of an lipid-containing particle, such as an LNP, as described herein that, when administered to a subject having a disease, can be sufficient to effect treatment of the disease (e.g., by diminishing, ameliorating or maintaining the existing disease or one or more symptoms of disease). The “therapeutically effective amount” may vary depending on the lipid-containing particle, such as an LNP, how the composition is administered, the disease and its severity, and the history, age, weight, family history, genetic makeup, the types of preceding or concomitant treatments, if any, and other individual characteristics of the subject to be treated.

A “therapeutically-effective amount” can also include an amount of an agent that produces a local or systemic effect at a reasonable benefit/risk ratio applicable to any treatment. Lipid-containing particle, such as an LNP, employed in the methods described herein may be administered in a sufficient amount to produce a reasonable benefit/risk ratio applicable to such treatment.

The phrase “pharmaceutically-acceptable carrier” as used herein can refer to a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier can be “acceptable” in the sense of being compatible with the other ingredients of the formulation and not injurious to the subject being treated. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (24) other non-toxic compatible substances employed in pharmaceutical formulations.

A “group” or “functional group” is a portion of a larger molecule comprising or consisting of a grouping of atoms and/or bonds that confer a chemical or physical quality to a molecule. A “residue” is the portion of a compound or monomer that remains in a larger molecule, such as a polymer chain, after incorporation of that compound or monomer into the larger molecule. A “moiety” is a portion of a molecule, and can comprise one or more functional groups, and in the case of an “active moiety” can be a characteristic portion of a molecule or compound that imparts activity, such as pharmacological or physiological activity, to a molecule as contrasted to inactive portions of a molecule such as esters of active moieties, or salts of active agents.

As used herein, the term “polymer composition” is a composition comprising one or more polymers. As a class, “polymers” includes, without limitation, homopolymers, heteropolymers, copolymers, block polymers, block co-polymers and can be both natural and synthetic. Homopolymers contain one type of building block, or monomer, whereas copolymers contain more than one type of monomer.

A polymer “comprises” or is “derived from” a stated monomer if that monomer is incorporated into the polymer. Thus, the incorporated monomer that the polymer comprises is not the same as the monomer prior to incorporation into the polymer, in that at the very least, during incorporation of the monomer, certain groups, e.g., terminal groups, that are modified during polymerization are changed, removed, and/or relocated, and certain bonds may be added, removed, and/or modified. A polymer is said to comprise a specific type of linkage if that linkage is present in the polymer. Unless otherwise specified, molecular weight for polymer compositions refers to weight average molecular weight (Mw).

As used herein, “alkyl” refers to straight, branched chain, or cyclic hydrocarbon groups including, for example, from 1 to about 20 carbon atoms, for example and without limitation C₁₋₃, C₁₋₆, C₁₋₁₀ groups, for example and without limitation, straight, branched chain alkyl groups such as methyl, ethyl, propyl, butyl, pentyl, hexyl, heptyl, octyl, nonyl, decyl, undecyl, dodecyl, and the like. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. “Lower alkyl” refers to C₁-C₆ alkyl. Non-limiting examples of straight alkyl groups include methyl, ethyl, propyl, butyl, pentyl, hexyl, heptyl, octyl, nonyl, and decyl. Branched alkyl groups comprises any straight alkyl group substituted with any number of alkyl groups. Non-limiting examples of branched alkyl groups include isopropyl, n-butyl, isobutyl, sec-butyl, and t-butyl. Non-limiting examples of cyclic alkyl groups include cyclopropyl, cyclobutyl, cyclopentyl, cyclohexyl, cycloheptlyl, and cyclooctyl groups. Cyclic alkyl groups also comprise fused-, bridged-, and spiro-bicycles and higher fused-, bridged-, and spiro-systems. A cyclic alkyl group can be substituted with any number of straight, branched, or cyclic alkyl groups. “Unsaturated alkyl” may comprise one or more, e.g., 1, 2, 3, 4, or 5, carbon-to-carbon double bonds and alternatively may be referred to as alkene or alkenyl, as described below. “Substituted alkyl” can include alkyl substituted at 1 or more (e.g., 1, 2, 3, 4, 5, 6, or more) positions, which substituents are attached at any available atom to produce a stable compound, with substitution as described herein. “Optionally substituted alkyl” refers to alkyl or substituted alkyl. “Halogen,” “halide,” and “halo” refers to —F, —Cl, —Br, and/or —I. “Alkylene” and “substituted alkylene” can include divalent alkyl and divalent substituted alkyl, respectively, including, without limitation, methylene, ethylene, trimethylene, tetramethylene, pentamethylene, hexamethylene, hepamethylene, octamethylene, nonamethylene, or decamethylene. “Optionally substituted alkylene” can include alkylene or substituted alkylene.

“Alkene or alkenyl” can include straight, branched chain, or cyclic hydrocarbyl groups including, e.g., from 2 to about 20 carbon atoms, such as, without limitation C6-24 groups in the case of fatty acids, having one or more, e.g., 1, 2, 3, 4, or 5, carbon-to-carbon double bonds, and may be referred to as “unsaturated alkyl” in the context of fatty acids an lipids. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. A halo-alkenyl group can be any alkenyl group substituted with any number of halogen atoms. “Substituted alkene” can include alkene substituted at 1 or more, e.g., 1, 2, 3, 4, or 5 positions, which substituents are attached at any available atom to produce a stable compound, with substitution as described herein. “Optionally substituted alkene” can include alkene or substituted alkene. Likewise, “alkenylene” can refer to divalent alkene. Examples of alkenylene include without limitation, ethenylene (—CH═CH—) and all stereoisomeric and conformational isomeric forms thereof. “Substituted alkenylene” can refer to divalent substituted alkene. “Optionally substituted alkenylene” can refer to alkenylene or substituted alkenylene.

An “ester” is represented by the formula —OC(O)R, where R can be an alkyl, alkenyl, or group described above.

Alkyne or “alkynyl” refers to a straight, branched chain, or cyclic unsaturated hydrocarbon having the indicated number of carbon atoms and at least one triple bond. The triple bond of an alkyne or alkynyl group can be internal or terminal. Examples of a (C₂-C₈)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein below. An alkyne or alkynyl group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. A halo-alkynyl group can be any alkynyl group substituted with any number of halogen atoms. The term “alkynylene” refers to divalent alkyne. Examples of alkynylene include without limitation, ethynylene, propynylene. “Substituted alkynylene” refers to divalent substituted alkyne.

“PEG” refers to polyethylene glycol. “PEGylated” refers to a compound comprising a moiety, comprising two or more consecutive ethylene glycol moieties. Non-limiting examples of PEG moieties for PEGylation of a compound include, one or more blocks of from 1 to 200 ethylene glycol units, such as —(O—CH₂—CH₂)ₙ—, —(CH₂—CH₂—O)ₙ—, or —(O—CH₂—CH₂)ₙ—OH, where n ranges, for example and without limitation, from 1 to 200 or from 1 to 100, for example from 1 to 5, or 1.

“Aryl,” alone or in combination refers to an aromatic ring system such as phenyl or naphthyl. “Aryl” also can include aromatic ring systems that are optionally fused with a cycloalkyl ring. A “substituted aryl” is an aryl that is independently substituted with one or more substituents attached at any available atom to produce a stable compound, wherein the substituents are as described herein. The substituents can be, for example, hydrocarbyl groups, alkyl groups, alkoxy groups, and halogen atoms. “Optionally substituted aryl” refers to aryl or substituted aryl. An aryloxy group can be, for example, an oxygen atom substituted with any aryl group, such as phenoxy. An arylalkoxy group can be, for example, an oxygen atom substituted with any aralkyl group, such as benzyloxy. “Arylene” denotes divalent aryl, and “substituted arylene” refers to divalent substituted aryl. “Optionally substituted arylene” refers to arylene or substituted arylene. A “polycyclic aryl group” and related terms, such as “polycyclic aromatic group” refers to a group composed of at least two fused aromatic rings. “Heteroaryl” or “hetero-substituted aryl” refers to an aryl group substituted with one or more heteroatoms, such as N, O, P, and/or S. Examples of heteroaryl groups include, but are not limited to, thienyl, furyl, pyridyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl.

“Cycloalkyl” refers to monocyclic, bicyclic, tricyclic, or polycyclic, 3- to 14-membered ring systems, which are either saturated, or partially unsaturated. The cycloalkyl group may be attached via any atom. Cycloalkyl also contemplates fused rings wherein the cycloalkyl is fused to an aryl or hetroaryl ring. Representative examples of cycloalkyl include, but are not limited to cyclopropyl, cyclobutyl, cyclopentyl, and cyclohexyl. A cycloalkyl group can be unsubstituted or optionally substituted with one or more substituents as described herein below. “Cycloalkylene” refers to divalent cycloalkyl. The term “optionally substituted cycloalkylene” refers to cycloalkylene that is substituted with at least 1, 2 or 3 substituents, attached at any available atom to produce a stable compound, wherein the substituents are as described herein.

“Carboxyl” or “carboxylic” refers to group having an indicated number of carbon atoms, where indicated, and terminating in a —C(O)OH group, thus having the structure —R—C(O)OH, where R is an unsubstituted or substituted divalent organic group that can include linear, branched, or cyclic hydrocarbons. Non-limiting examples of these include: C₁₋₈ carboxylic groups, such as ethanoic, propanoic, 2-methylpropanoic, butanoic, 2,2-dimethylpropanoic, pentanoic, etc. “Amine” or “amino” refers to group having the indicated number of carbon atoms, where indicated, and terminating in a —NH₂ group, thus having the structure —R—NH₂, where R is a unsubstituted or substituted divalent organic group that, e.g., includes linear, branched, or cyclic hydrocarbons, and optionally comprises one or more heteroatoms. The term “alkylamino” refers to a radical of the formula —NHR^{x} or —NR^{x}R^{x} where each R^{x} is, independently, an alkyl radical as defined above.

Terms combining the foregoing refer to any suitable combination of the foregoing, such as arylalkenyl, arylalkynyl, heteroarylalkyl, heteroarylalkenyl, heteroarylalkynyl, heterocyclylalkyl, heterocyclylalkenyl, heterocyclylalkynyl, heteroaryl, heterocyclyl, cycloalkyl, cycloalkenyl, alkylarylalkyl, alkylarylalkenyl, alkenylarylalkynyl, alkylarylalkynyl, alkenylarylalkyl, alkenylarylalkenyl, alkylheteroarylalkyl, alkynylarylalkyl, alkynylarylalkenyl, alkynylarylalkynyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, and alkynylhereroaryl. As an example, “arylalkylene” refers to a divalent alkylene wherein one or more hydrogen atoms in an alkylene group is replaced by an aryl group, such as a (C₃-C₈)aryl group. Examples of (C₃-C₈)aryl-(C₁-C₆)alkylene groups include without limitation 1-phenylbutylene, phenyl-2-butylene, I-phenyl-2-methylpropylene, phenylmethylene, phenylpropylene, and naphthylethylene. The term “(C₃-C₈)cycloalkyl-(C₁-C₆)alkylene” refers to a divalent alkylene wherein one or more hydrogen atoms in the C₁-C₆ alkylene group is replaced by a (C₃-C₈)cycloalkyl group. Examples of (C₃-C₅)cycloalkyl-(C₁-C₆)alkylene groups include without limitation 1-cycloproylbutylene, cycloproyl-2-butylene, cyclopentyl-1-phenyl-2-methylpropylene, cyclobutylmethylene and cyclohexylpropylene.

A fatty acid is an aliphatic monocarboxylic acid, comprising a carboxyl group linked to an aliphatic hydrocarbyl group which may be saturated or unsaturated. A hydrocarbyl or hydrocarbon group refers to a group of carbon and hydrogen atoms, such as alkyl, alkenyl (alternatively, unsaturated alkyl), or aryl groups. By “aliphatic”, it is meant acyclic or cyclic, saturated or unsaturated hydrocarbon compounds, excluding aromatic compounds. The aliphatic group of fatty acids is typically a linear chain of carbons, but fatty acids and substituted fatty acids as a class include linear, branched, and/or cyclic carbon chains. As used herein, fatty acids include both natural and synthetic aliphatic carboxylic acids. Fatty acids can have an aliphatic chain of from three to 40 carbon atoms (for example, as used herein, “a (C₃-C₄₀) fatty acid”). Hydrogen atoms of a compound, such as a fatty acid may be substituted with a group or moiety (hereinafter referred to as a “substituent”), to produce a substituted fatty acid. Fatty acids and substituted fatty acids may be referred to as “optionally substituted fatty acids”) Fatty acids, and fatty acid groups, may be referred to by the number of carbon atoms and the number of double bonds, e.g., C10:0, referring to a fatty acid or fatty acid group having 10 carbon atoms and zero double bonds. Likewise, C18:1 refers to a fatty acid with an 18-carbon chain having one double bond, such as oleic acid.

Unsaturated fatty acids and substituted unsaturated fatty acids (collectively “optionally substituted unsaturated fatty acids”) comprise one or more carbon-carbon double bonds, or an alkenyl group (e.g., vinyl group) in their aliphatic chains. The individual carbon atoms of the alkenyl group are referred to herein as alkenyl carbons. Unless specified, any carbon-carbon double bond in the alkyl chain of the described optionally substituted unsaturated fatty acids independently may be E (trans) or Z (cis) geometric isomers, or mixtures thereof.

Fatty acids may include, without limitation: C3, C4, C5, C6, C7, C8, C9, C10, C11, C12, C13, C14, C15, C16, C17, C18, C19, C20, C21, C22, C23, C24, C25, C26, C27, C28, C29, C30, C31, C32, C33, C34, C35, C36, C37, C38, C39, and C40 fatty acids. The fatty acids may be saturated (zero double bonds), or unsaturated, e.g., with 0 or 1, 2, 3, 4, 5, 6, or more double bonds. Non-limiting examples of saturated fatty acids include: propionic acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, carboceric acid, montanic acid, nonacosylic acid, melissic acid, hentriacontylic acid, lacceroic acid, psyllic acid, geddic acid, ceroplastic acid, hexatriacontylic acid, heptatriacontylic acid, octatriacontylic acid, nonatriacontylic acid, and tetracontylic acid. Non-limiting examples of unsaturated fatty acids include: crotonic acid, myristoleic acid, palmitoleic acid, sapienic acid, oleic acid, elaidic acid, vaccenic acid, gadoleic acid, eicosenoic acid, erucic acid, nervonic acid, linoleic acid, eicosadienoic acid, docosadienoic acid, linolenic acid, pinolenic acid, eleostearic acid, mead acid, dihomo-γ-linolenic acid, eicosatrienoic acid, stearidonic acid, arachidonic acid, eicosatetraenoic acid, adrenic acid, bosseopentaenoic acid, eicosapentaenoic acid, ozubondo acid, sardine acid, tetracosanolpentaenoic acid, cervonic acid, and herring acid.

Compounds described herein, including fatty acids and substituted fatty acids can exist in various isomeric forms, including configurational, geometric, and conformational isomers, as well as existing in various tautomeric forms, such as those that differ in the point of attachment of a hydrogen atom. The term “isomer” is intended to encompass all isomeric forms of a compound of this invention, including tautomeric forms of the compound.

Certain compounds described here may have asymmetric centers and therefore exist in different enantiomeric and diastereomeric forms. A compound can be in the form of an optical isomer or a diastereomer. Accordingly, compounds described herein include their optical isomers, diastereoisomers and mixtures thereof, including a racemic mixture unless otherwise specified. Optical isomers of the compounds of the invention can be obtained by known techniques such as asymmetric synthesis, chiral chromatography, simulated moving bed technology, or via chemical separation of stereoisomers through the employment of optically active resolving agents.

Unless otherwise indicated, “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

Lipids, as a group, include glycerides and phospholipids. A “glyceride” is an ester of glycerol (propane 1,2,3-triol) with a fatty acid or a substituted fatty acid. Phospholipids are lipids containing phosphoric acid as mono- or di-esters, such as phosphatidic acids and phosphoglycerides. Phosphoglycerides are di-esters of glycerol, which are glycerol derivatives in which one hydroxyl group of the glycerol is phosphodiester-linked to a group, such as a functional group, such as, for example and without limitation, a 2-amino ethanol or a choline (e.g., —O—CH₂—CH₂—N⁺(CH₃)₃) groups. A phosphatidylcholine is a phosphoglyceride with a choline linked to the glycerol moiety by a phosphodiester linkage. A glycerol-phosphoethanolamine is a phosphoglyceride with an 2-amino ethane group (e.g., —CH₂—CH₂—NH₃) linked to the glycerol moiety by a phosphodiester linkage. Amphipathic refers to a molecule or compound having both hydrophobic and hydrophilic parts, e.g., under physiological conditions.

Provided herein is a lipid-containing particle, e.g., a lipid nanoparticle or microparticle, formulation for CNS delivery. Lipid nanoparticles incorporate sphingolipid helper lipids that aid the nanoparticles in delivery to cells, tissue and organs of the central nervous system when delivered intrathecally, intracranially, or to the base of the skull, e.g., into cerebrospinal fluid of the spinal cord, brain, or brainstem, via intra-cerebral ventricular injection, and/or to the brainstem. The sphingolipids when used as helper lipids in lipid particles as described herein, provide superior delivery to cells of the central nervous system as compared to previously-described lipid nanoparticles.

Sphingolipids are lipids that include a sphingoid base, an optional fatty acid, and an optional a head group. The sphingoid base comprises an unsaturated or saturated chain of carbon atoms (e.g., 18 carbon atoms) with one to three hydroxyl groups and one amino group at position 2 of the carbon chain. A generic structure of a sphingolipid (not including stereochemistry) may be as follows:

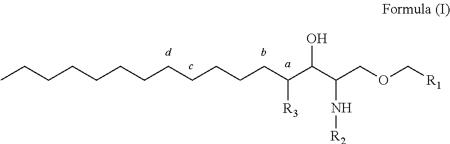

where:
R₁ is hydrogen or the optional head group;
R₂ is hydrogen or a fatty acid linked via an amide bond; and
R₃ is hydrogen or a hydroxyl group,
   wherein when R₃ is hydrogen, the carbon-carbon bond between a and b may be a single bond or a double bond and the carbon-carbon bond between c and d may be a single bond or a double bond,
   wherein when R₃ is a hydroxyl group, the carbon-carbon bond between a and b may be a single bond and the carbon-carbon bond between c and d may be a single bond or a double bond.

For example, the sphingolipid of Formula (I) may have sphingosine as the sphingoid base, where R₃ is hydrogen, the carbon-carbon bond between a and b is a double bond, and the carbon-carbon bond between c and d is a single bond:

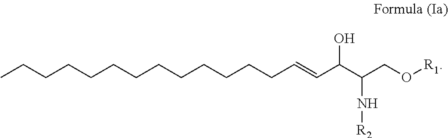

The sphingolipid of Formula (I) may have dihydrosphingosine as the sphingoid base, where R₃ is hydrogen, the carbon-carbon bond between a and b is a single bond, and the carbon-carbon bond between c and d is a single bond:

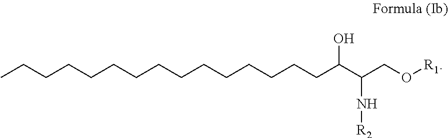

The sphingolipid of Formula (I) may have phytosphingosine as the sphingoid base, where R₃ is a hydroxyl group, the carbon-carbon bond between a and b is a single bond, and the carbon-carbon bond between c and d is a single bond:

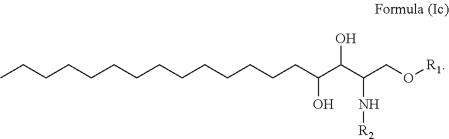

The sphingolipid of Formula (I) may have dihydrophytosphingosine as the sphingoid base, where R₃ is a hydroxyl group, the carbon-carbon bond between a and b is a single bond, and the carbon-carbon bond between c and d is a double bond:

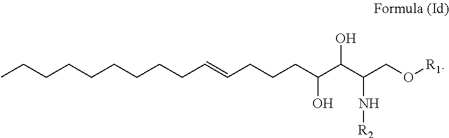

When R₁ is a head group in Formula (I), the head group may be linked to the oxygen atom via a phosphodiester linkage or via a glycosidic linkage. The head group may comprise phosphocholine or phosphoethanolamine, where the phosphocholine or phosphoethanolamine may be linked to the oxygen atom of Formula (I) via a phosphodiester linkage. Alternatively, the head group may comprise a carbohydrate and may be linked to the oxygen atom via a glycosidic bond. As used herein, “carbohydrate” includes one or more monosaccharides that are linked together by glycosidic bonds. The carbohydrate may include a monosaccharide. Alternatively, the carbohydrate may include two monosaccharides (a disaccharide), where the two monosaccharides are linked together via a glycosidic bond. Alternatively, the carbohydrate may include three to ten monosaccharides (an oligosaccharide) or the carbohydrate may include greater than ten monosaccharides (a polysaccharide), where each monosaccharide is linked to the next monosaccharide via a glycosidic bond. The carbohydrate may be a linear chain of monosaccharides or may be a branched chain of monosaccharides. The monosaccharides may optionally comprise a sulfonate group, an amide group, a carboxylic acid group, a sialic acid group, or a combination thereof.

When R₂ is a fatty acid chain in Formula (I), the acyl group of the fatty acid is linked to the sphingoid base via an amide bond.

Non-limiting examples of suitable sphingolipids include ceramides, sphingomyelins, ceramide phosphoethanolamine, sphingosylphosphorylcholine, sphingosine, glycosphingolipids, and combinations thereof.

The sphingolipid may be ceramide. A ceramide comprises the generic structure of formula (I), where R₁ is hydrogen and R₂ is a fatty acid linked via an amide bond.

For example, the ceramide may comprise the following generic structure:

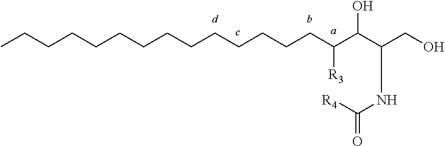

Formula (II),
where R₃ may hydrogen or a hydroxyl group,
   wherein when R₃ is hydrogen, the carbon-carbon bond between a and b may be a single bond or a double bond and the carbon-carbon bond between c and d may be a single bond or a double bond,
   wherein when R₃ is a hydroxyl group, the carbon-carbon bond between a and b may be a single bond and the carbon-carbon bond between c and d may be a single bond or a double bond, and
where R₄ comprises a saturated fatty acid chain, a monounsaturated fatty acid chain, a polyunsaturated fatty acid chain, an omega hydroxy saturated fatty acid chain, an omega hydroxy monounsaturated fatty acid chain, or an omega hydroxy polyunsaturated fatty acid chain comprising from 1 to 31 carbon atoms, such as from 10 to 25 carbon atoms.

In the ceramide of Formula (II), when R₄ comprises a saturated fatty acid chain or an omega hydroxy saturated fatty acid chain, the chain may be CₙH₂ₙ₊₁, where n may range from 1 to 31 or 10 to 25, inclusive. For example, the saturated fatty acid chain may be CH₃, C₃H₇, C₅H₁₁, C₇H₁₅, C₉H₁₉, C₁₁H₂₃, C₁₃H₂₇, C₁₅H₃₁, C₁₇H₃₅, C₁₉H₃₉, C₂₁H₄₃, C₂₃H₄₇, C₂₅H₅₁, C₂₇H₅₅, C₂₉H₅₉, or C₃₁H₆₃.

In the ceramide of Formula (II), when R₄ comprises a monounsaturated fatty acid chain or an omega hydroxy monounsaturated fatty acid chain, the chain may be CₙH₂ₙ₋₁, where n may range from 10 to 25 or from 17 to 23, inclusive. For example, the monounsaturated fatty acid chain may be C₁₇H₃₃ or C₂₃H₄₅.

For example, the ceramide may comprise the following structure and is referred to herein as ceramide:

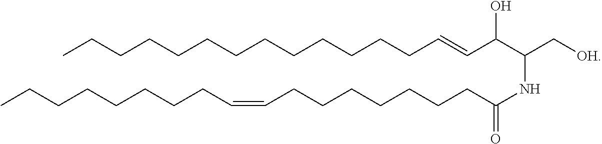 [SMILES: CCCCCCCC/C=C\CCCCCCCC(=O)NC(CO)C(O)/C=C/CCCCCCCCCCCCC]

The sphingolipid may be sphingomyelin. Sphingomyelins are phosphorus-containing sphingolipids and comprise the generic structure of Formula (I), where R₁ is a phosphocholine head group attached via a phosphodiester linkage and R₂ is a fatty acid linked via an amide bond.

For example, the sphingomyelin may comprise the following generic structure:

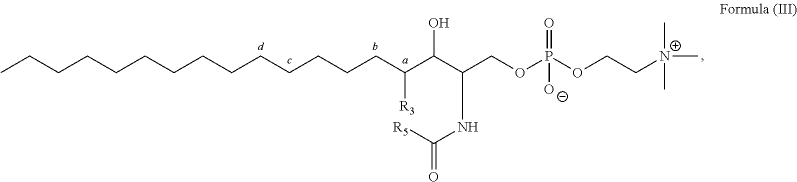

where R₃ may hydrogen or a hydroxyl group,
   wherein when R₃ is hydrogen, the carbon-carbon bond between a and b may be a single bond or a double bond and the carbon-carbon bond between c and d may be a single bond or a double bond,
   wherein when R₃ is a hydroxyl group, the carbon-carbon bond between a and b may be a single bond and the carbon-carbon bond between c and d may be a single bond or a double bond, and
where R₅ comprises a saturated fatty acid chain, a monounsaturated fatty acid chain, a polyunsaturated fatty acid chain, an omega hydroxy saturated fatty acid chain, an omega hydroxy monounsaturated fatty acid chain, or an omega hydroxy polyunsaturated fatty acid chain comprising from 1 to 31 carbon atoms, such as from 10 to 25 carbon atoms.

In the sphingomyelin of Formula (III), when R₅ comprises a saturated fatty acid chain or an omega hydroxy saturated fatty acid chain, the chain may be CₙH₂ₙ₊₁, where n may range from 1 to 31 or 10 to 25, inclusive. For example, the saturated fatty acid chain may be CH₃, C₃H₇, C₅H₁₁, C₇H₁₅, C₉H₁₉, C₁₁H₂₃, C₁₃H₂₇, C₁₅H₃₁, C₁₇H₃₅, C₁₉H₃₉, C₂₁H₄₃, C₂₃H₄₇, C₂₅H₅₁, C₂₇H₅₅, C₂₉H₅₉, or C₃₁H₆₃.

In the sphingomyelin of Formula (III), when R₅ comprises a monounsaturated fatty acid chain or an omega hydroxy monounsaturated fatty acid chain, the chain may be CₙH₂ₙ₋₁, where n may range from 10 to 25 or from 17 to 23, inclusive. For example, the monounsaturated fatty acid chain may be C₁₇H₃₃ or C₂₃H₄₅.

For example, the sphingomyelin may comprise the following structure:

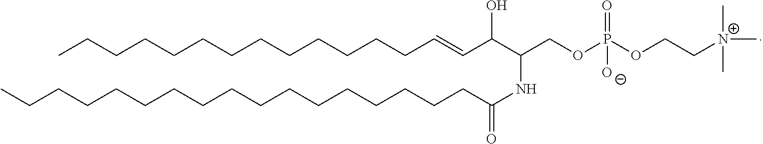 [SMILES: CCCCCCCCCCCCC/C=C/C(O)C(COP(=O)([O-])OCC[N+](C)(C)C)NC(=O)CCCCCCCCCCCCCCCCC]

The sphingolipid may be ceramide phosphoethanolamine. Ceramide phosphoethanolamine may comprise the generic structure of Formula (I), where R₁ is phosphoethanolamine and R₂ is a fatty acid linked via an amide bond.

For example, the ceramide phosphoethanolamine may comprise the following generic structure:

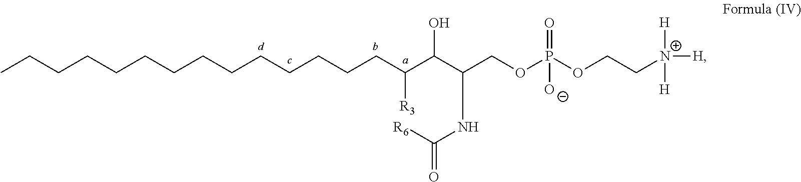

where R₃ may hydrogen or a hydroxyl group,
   wherein when R₃ is hydrogen, the carbon-carbon bond between a and b may be a single bond or a double bond and the carbon-carbon bond between c and d may be a single bond or a double bond,
   wherein when R₃ is a hydroxyl group, the carbon-carbon bond between a and b may be a single bond and the carbon-carbon bond between c and d may be a single bond or a double bond, and
where R₅ comprises a saturated fatty acid chain, a monounsaturated fatty acid chain, a polyunsaturated fatty acid chain, an omega hydroxy saturated fatty acid chain, an omega hydroxy monounsaturated fatty acid chain, or an omega hydroxy polyunsaturated fatty acid chain comprising from 1 to 31 carbon atoms, such as from 10 to 25 carbon atoms.

In the above ceramide phosphoethanolamine of Formula (IV), when R₆ comprises a saturated fatty acid chain or an omega hydroxy saturated fatty acid chain, the chain may be CₙH₂ₙ₊₁, where n may range from 1 to 27 or from 10 to 25, inclusive. For example, the saturated fatty acid chain may be CH₃, C₃H₇, C₅H₁₁, C₇H₁₅, C₉H₁₉, C₁₁H₂₃, C₁₃H₂₇, C₁₅H₃₁, C₁₆H₃₃, C₁₇H₃₅, C₁₉H₃₉, C₂₁H₄₃, C₂₂H₄₅, C₂₃H₄₇, C₂₅H₅₁, C₂₇H₅₅, C₂₉H₅₉, or C₃₁H₆₃.

In the above ceramide phosphoethanolamine of Formula (IV), when R₆ comprises a monounsaturated fatty acid chain or an omega hydroxy monounsaturated fatty acid chain, the chain may be CₙH₂ₙ₋₁, where n may range from 10 to 25 or from 17 to 23, inclusive. For example, the unsaturated fatty acid chain may be C₁₇H₃₃ or C₂₃H₄₅.

The sphingolipid may be sphingosylphosphorylcholine. Sphingosylpshphorylcholine comprises the following structure:

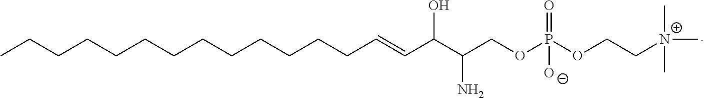 [SMILES: CCCCCCCCCCCCC/C=C/C(O)C(N)COP(=O)([O-])OCC[N+](C)(C)C]

The sphingolipid may be sphingosine. Sphingosine comprises the following structure:

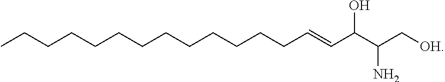 [SMILES: CCCCCCCCCCCCC/C=C/C(O)C(N)CO]

The sphingolipid may be a glycosphingolipid. Glycosphingolipids are carbohydrate containing sphingolipids and comprise the generic structure of formula (I), where R₁ is a carbohydrate and R₂ is a fatty acid linked via an amide bond.

The glycosphingolipid may be a glucosyl sphingolipid comprising the following generic structure:

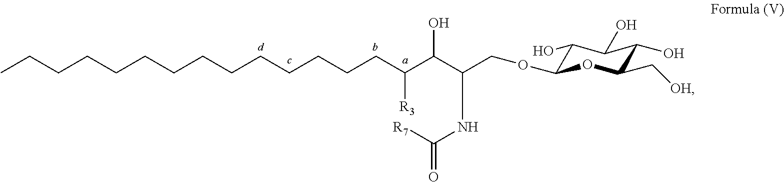

where R₃ may hydrogen or a hydroxyl group,
   wherein when R₃ is hydrogen, the carbon-carbon bond between a and b may be a single bond or a double bond and the carbon-carbon bond between c and d may be a single bond or a double bond,
      wherein when R₃ is a hydroxyl group, the carbon-carbon bond between a and b may be a single bond and the carbon-carbon bond between c and d may be a single bond or a double bond, and
where R₇ comprises a saturated fatty acid chain, a monounsaturated fatty acid chain, a polyunsaturated fatty acid chain, an omega hydroxy saturated fatty acid chain, an omega hydroxy monounsaturated fatty acid chain, or an omega hydroxy polyunsaturated fatty acid chain comprising from 1 to 31 carbon atoms, such as from 10 to 25 carbon atoms.

The glycosphingolipid may be a galactosyl sphingolipid comprising the following generic structure:

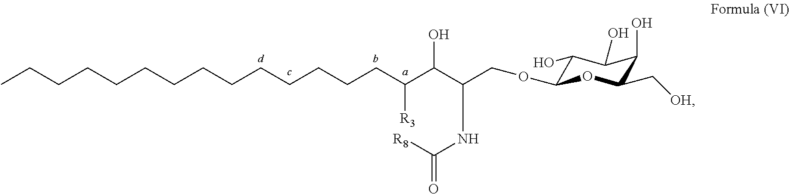

where R₃ may hydrogen or a hydroxyl group,
   wherein when R₃ is hydrogen, the carbon-carbon bond between a and b may be a single bond or a double bond and the carbon-carbon bond between c and d may be a single bond or a double bond,
   wherein when R₃ is a hydroxyl group, the carbon-carbon bond between a and b may be a single bond and the carbon-carbon bond between c and d may be a single bond or a double bond, and
where R₈ comprises a saturated fatty acid chain, a monounsaturated fatty acid chain, a polyunsaturated fatty acid chain, an omega hydroxy saturated fatty acid chain, an omega hydroxy monounsaturated fatty acid chain, or an omega hydroxy polyunsaturated fatty acid chain comprising from 1 to 31 carbon atoms, such as from 10 to 25 carbon atoms.

The glycosphingolipid may be a lactosyl sphingolipid comprising the following generic structure:

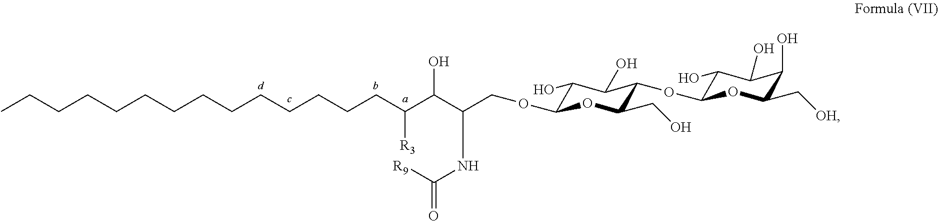

where R₃ may hydrogen or a hydroxyl group,
   wherein when R₃ is hydrogen, the carbon-carbon bond between a and b may be a single bond or a double bond and the carbon-carbon bond between c and d may be a single bond or a double bond,
   wherein when R₃ is a hydroxyl group, the carbon-carbon bond between a and b may be a single bond and the carbon-carbon bond between c and d may be a single bond or a double bond, and
where R₉ comprises a saturated fatty acid chain, a monounsaturated fatty acid chain, a polyunsaturated fatty acid chain, an omega hydroxy saturated fatty acid chain, an omega hydroxy monounsaturated fatty acid chain, or an omega hydroxy polyunsaturated fatty acid chain comprising from 1 to 31 carbon atoms, such as from 10 to 25 carbon atoms.

In the above glycosphingolipids of Formulas (V)-(VII), when R₇, R₈, or R₉ comprise a saturated fatty acid chain or an omega hydroxy saturated fatty acid chain, the chain may be CₙH₂ₙ₊₁, where n may range from 1 to 27 or from 10 to 25, inclusive. For example, the saturated fatty acid chain may be CH₃, C₃H₇, C₅H₁₁, C₇H₁₅, C₉H₁₉, C₁₁H₂₃, C₁₃H₂₇, C₁₅H₃₁, C₁₆H₃₃, C₁₇H₃₅, C₁₉H₃₉, C₂₁H₄₃, C₂₂H₄₅, C₂₃H₄₇, C₂₅H₅₁, C₂₇H₅₅, C₂₉H₅₉, or C₃₁H₆₃.

In the above glycosphingolipids of Formulas (V)-(VII), when R₇, R₈, or R₉ comprise a monounsaturated fatty acid chain or an omega hydroxy monounsaturated fatty acid chain, the chain may be CₙH₂ₙ₋₁, where n may range from 10 to 25 or from 17 to 23, inclusive. For example, the monounsaturated fatty acid chain may be C₁₇H₃₃ or C₂₃H₄₅.

For example, the glucosyl glycosphingolipid may comprise the following structure and is referred to herein as glucosyl ceramide:

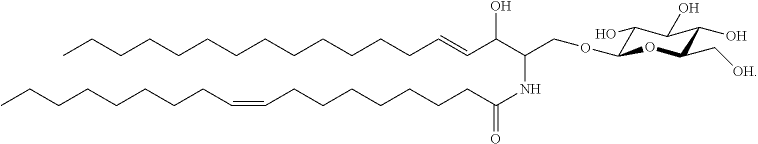

For example, the galactosyl glycosphingolipid may comprise the following structure and is referred to herein as galactosyl ceramide:

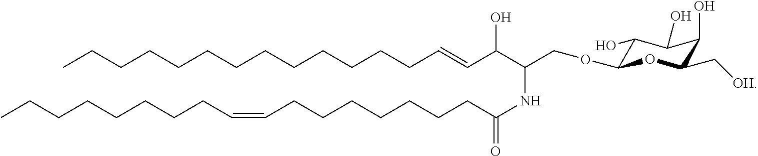

For example, the lactosyl glycosphingolipid may comprise the following structure and is referred to herein as lactosyl ceramide:

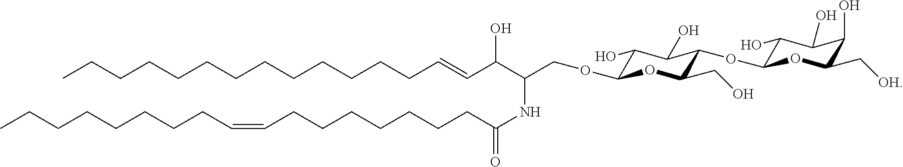

For example, the glycosphingolipid may be a sulfatide comprising the following generic structure:

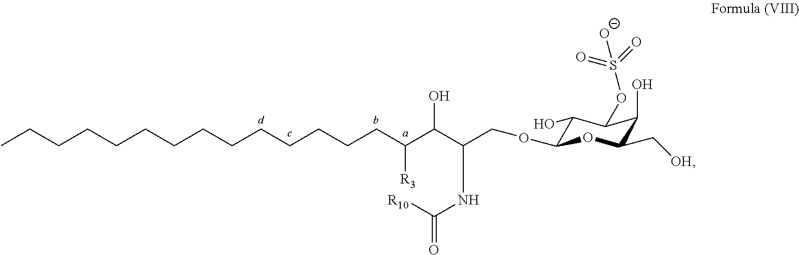

where R₃ may hydrogen or a hydroxyl group,
   wherein when R₃ is hydrogen, the carbon-carbon bond between a and b may be a single bond or a double bond and the carbon-carbon bond between c and d may be a single bond or a double bond,
   wherein when R₃ is a hydroxyl group, the carbon-carbon bond between a and b may be a single bond and the carbon-carbon bond between c and d may be a single bond or a double bond, and
where R₁₀ comprises a saturated fatty acid chain, a monounsaturated fatty acid chain, a polyunsaturated fatty acid chain, an omega hydroxy saturated fatty acid chain, an omega hydroxy monounsaturated fatty acid chain, or an omega hydroxy polyunsaturated fatty acid chain comprising from 1 to 31 carbon atoms, such as from 10 to 25 carbon atoms.

In the above sulfatide of Formula (VIII), when R₁₀ comprises a saturated fatty acid chain or an omega hydroxy saturated fatty acid chain, the chain may be CₙH₂ₙ₊₁, where n may range from 1 to 27 or from 10 to 25, inclusive. For example, the saturated fatty acid chain may be CH₃, C₃H₇, C₅H₁₁, C₇H₁₅, C₉H₁₉, C₁₁H₂₃, C₁₃H₂₇, C₁₅H₃₁, C₁₆H₃₃, C₁₇H₃₅, C₁₉H₃₉, C₂₁H₄₃, C₂₂H₄₅, C₂₃H₄₇, C₂₅H₅₁, C₂₇H₅₅, C₂₉H₅₉, or C₃₁H₆₃.

In the above sulfatide of Formula (VIII), when R₁₀ comprises a monounsaturated fatty acid chain or an omega hydroxy monounsaturated fatty acid chain, the chain may be CₙH₂ₙ₋₁, where n may range from 10 to 25 or from 17 to 23, inclusive. For example, the monounsaturated fatty acid chain may be C₁₇H₃₃ or C₂₃H₄₅.

For example, the sulfatide may be a brain sulfatide comprising the following structure:

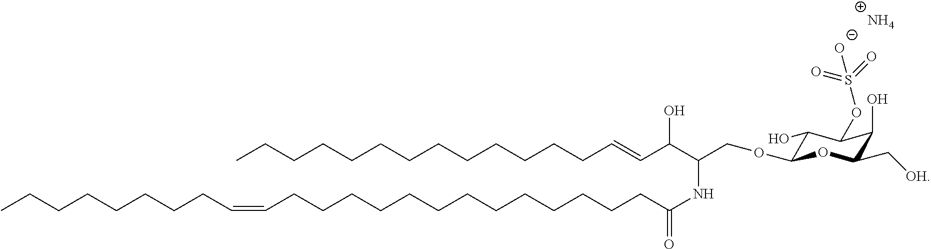

The glycosphingolipid may be glucosyl sphingosine comprising the following generic structure:

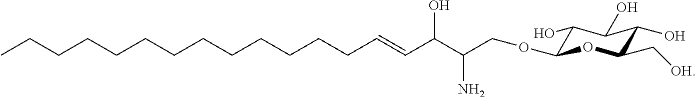

The glycosphingolipid may be galactosyl sphingosine comprising the following generic structure:

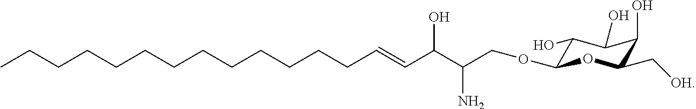

The glycosphingolipid may be lactosyl sphingosine comprising the following generic structure:

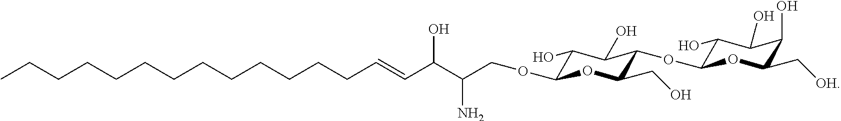

The glycosphingolipid may be a ganglioside comprising the following generic structure:

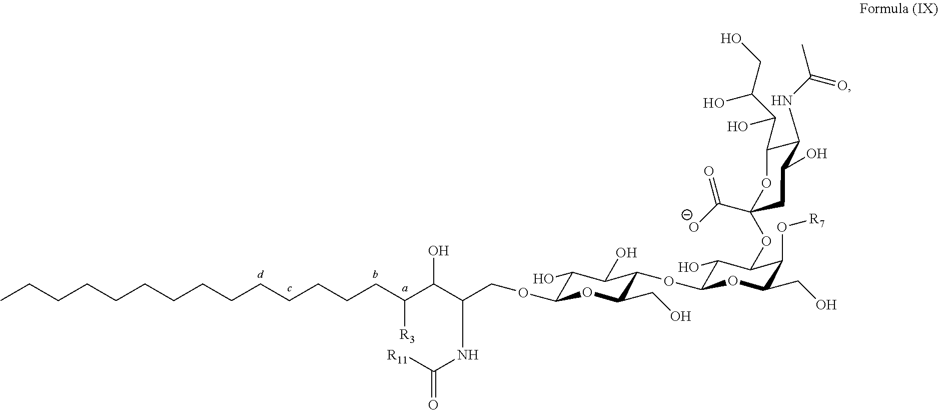

where R₃ may hydrogen or a hydroxyl group,
   wherein when R₃ is hydrogen, the carbon-carbon bond between a and b may be a single bond or a double bond and the carbon-carbon bond between c and d may be a single bond or a double bond,
   wherein when R₃ is a hydroxyl group, the carbon-carbon bond between a and b may be a single bond and the carbon-carbon bond between c and d may be a single bond or a double bond, and
where R₁₁ comprises a saturated fatty acid chain, a monounsaturated fatty acid chain, a polyunsaturated fatty acid chain, an omega hydroxy saturated fatty acid chain, an omega hydroxy monounsaturated fatty acid chain, or an omega hydroxy polyunsaturated fatty acid chain comprising from 1 to 31 carbon atoms, such as from 10 to 25 carbon atoms, and R₇ comprises a hydrogen atom,

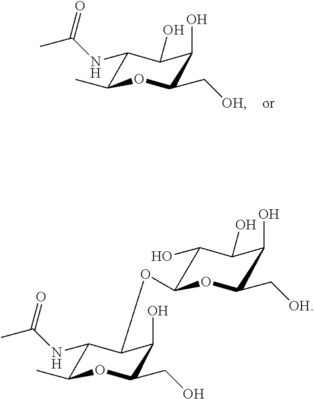

In the above ganglioside of Formula (IX), when R₁₁ comprises a saturated fatty acid chain or an omega hydroxy saturated fatty acid chain, the chain may be CₙH₂ₙ₊₁, where n may range from 1 to 27 or from 10 to 25, inclusive. For example, the saturated fatty acid chain may be CH₃, C₃H₇, C₅H₁₁, C₇H₁₅, C₉H₁₉, C₁₁H₂₃, C₁₃H₂₇, C₁₅H₃₁, C₁₆H₃₃, C₁₇H₃₅, C₁₉H₃₉, C₂₁H₄₃, C₂₂H₄₅, C₂₃H₄₇, C₂₅H₅₁, C₂₇H₅₅, C₂₉H₅₉, or C₃₁H₆₃.

In the above ganglioside of Formula (IX), when R₁₁ comprises a monounsaturated fatty acid chain or an omega hydroxy monounsaturated fatty acid chain, the chain may be CₙH₂ₙ₋₁, where n may range from 10 to 25 or from 17 to 23, inclusive. For example, the monounsaturated fatty acid chain may be C₁₇H₃₃ or C₂₃H₄₅.

For example, the ganglioside may be a brain ganglioside comprise the following structure:

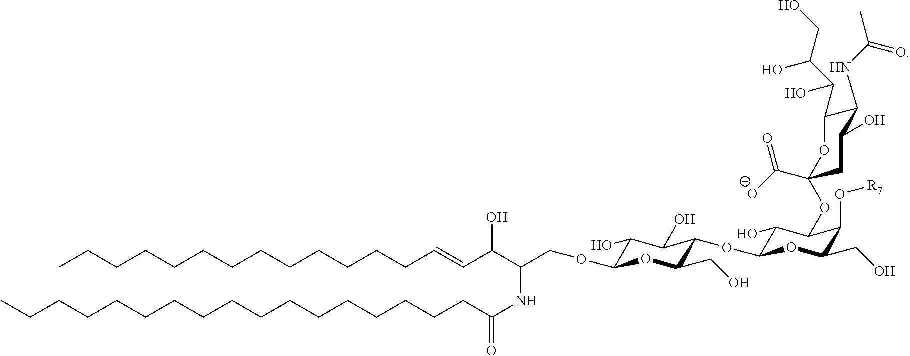

The sphingolipid helper lipid may be a sphingolipid may be a sphingolipid naturally found in brain and/or CNS tissue, such as vertebrate, mammalian, or human CNS or brain tissue, referred to herein as a “CNS sphingolipid” or a “brain sphingolipid”. Non-limiting examples of brain sphingolipids include: sphingomyelin (Brain SM), brain ganglioside, and brain sulfatide.

Lipid-containing particles, such as lipid nanoparticles are provided that provide superior delivery to cells of the CNS. Examples of lipid-containing particles, as described herein comprise, without limitation:
a helper sphingolipid;
cholesterol or a derivative thereof;
a PEG-based compound, such as a PEG-containing polymer or a PEGylated fatty acid-containing compound; and
an ionizable lipid (lipidoid).

The lipid-containing particles may be described as lipid nanoparticles or lipid microparticles, depending on their size. The particles may be used to deliver any compatible cargo or active agent, such as, without limitation, a polynucleotide, a drug, a protein or peptide, a small molecule, or a gas. The particles may be used to deliver an anionic or polyanionic cargo to cells, tissue and/or organs of a patient's central nervous system. The anionic or polyanionic cargo may be a protein or a peptide. The anionic or polyanionic cargo may be a nucleic acid, such as, without limitation: an mRNA, an antisense reagent, an RNAi agent, RNA or DNA encoding genome editing RNAs such as a guide RNA (gRNA) or prime editing RNA (pegRNA) and an mRNA encoding Cas9 or a Cas9 fusion protein, a genetic vector or recombinant construct such as a plasmid or other extrachromosomal or chromosome-targeting nucleic acid, a recombinant or natural viral genome, DNA comprising a gene, a ribozyme, or an aptamer. For example and without limitation, the agent or cargo may be an RNA (e.g., mRNA, an RNAi reagent, a dsRNA, an siRNA, an shRNA, an miRNA, an antisense RNA, a guide RNA (gRNA), a long non-coding RNA (lncRNA), a base editing gRNA (beRNA), a prime editing gRNA (pegRNA), or a transfer RNA (tRNA)). The cargo may be an mRNA, e.g., a capped and optionally PEGylated mRNA, encoding a therapeutic polypeptide or protein, or an immunogen, or may be non-coding.

The lipid particles described herein may also be incorporated into drug delivery devices, e.g., drug products, dosage forms, unit dosage forms, etc. the lipid particles may be used to encapsulate agents including polynucleotides, small molecules, proteins, peptides, metals, organometallic compounds, etc.

“Nucleic acids” include DNA and RNA as is found naturally, and chemically-modified nucleic acids, as are broadly-known, but optionally may not contain, as a class, peptide-nucleic acids (PNAs) having a neutral backbone, though modified peptide nucleic acids that are modified with anionic moieties, such as gamma-modified PNAs, may find use in the present compositions and methods. Nucleic acids useful in the compositions and methods described herein may be polyanionic nucleic acids, having an overall negative charge under neutral or physiological conditions, such as in an aqueous solution pH 6-8, e.g., in water, blood, serum, Ringer's, or normal saline. A nucleic acid may comprises a phosphorus-containing moiety, such as a phosphate and/or a phosphorothioate moiety, and therefore would be polyanionic. Non-limiting examples of nucleic acids include RNAi agents, antisense reagents, aptamers, and ribozymes, among others (see, e.g., Bajan S, Hutvagner G. RNA-Based Therapeutics: From Antisense Oligonucleotides to miRNAs. Cells. 2020 Jan. 7; 9(1):137; Invitrogen RNAi Handbook, ThermoFisher Scientific 2015; and Kilanowska, A., et al., In vivo and in vitro studies of antisense oligonucleotides—a review, RSC Adv., 2020, 10, 34501). Nucleic acids may be unmodified (e.g., natural) or chemically-modified (see, e.g., Dar, S., et al., siRNAmod: A database of experimentally validated chemically modified siRNAs. Sci Rep 6, 20031 (2016) and crdd.osdd.net/servers/sirnamod/).

The diameter of the lipid-containing particles may range from 1 micrometer to 1,000 micrometers (microparticles). The diameter of the particles range may range from 1 micrometer to 100 micrometers, from 1 micrometer to 10 micrometers, from 10 micrometers to 100 micrometers, from 100 micrometer to 1,000 micrometers, or from 1-5 micrometers. The diameter of the lipid particles may range from between 1 nm to 1,000 nm (nanoparticles), from 1 nm to 100 nm, from 1 nm to 10 nm, from 10 nm to100 nm, from 100 nm to 1,000 nm, from 20 nm to 2,000 nm, or from 1 to 5 nm. The diameter of the particles range from between 1 pm to 1,000 pm, from 1 pm to 100 pm, from 1 pm to 10 pm, from 10 pm to 100 pm, from 100 pm to 1,000 pm, or from 1 to 5 pm.

The lipid particles may be prepared using any useful method. These include, but are not limited to, spray drying, single and double emulsion solvent evaporation, solvent extraction, phase separation, and simple and complex coacervation, among other methods. The method of preparing the particles may be the double emulsion process and spray drying. The conditions used in preparing the particles may be altered to yield particles of a desired size or property (e.g., hydrophobicity, hydrophilicity, external morphology, “stickiness”, shape, etc.). The method of preparing the particle and the conditions (e.g., solvent, temperature, concentration, air flow rate, etc.) used may also depend on the agent being encapsulated and/or the composition of the matrix. Methods developed for making particles for delivery of encapsulated agents are amply described in the literature. In one example, the lipid-containing particles are prepared by microfluidics (see, e.g., Chen D, et al., Rapid discovery of potent siRNA-containing lipid nanoparticles enabled by controlled microfluidic formulation. J Am Chem Soc. 2012 Apr. 25; 134 (16):6948-51 and Cayabyab, C, et al., “mRNA Lipid Nanoparticles: Robust low-volume production for screening high-value nanoparticle materials,” Document ID: mrnaspark-AN-1018, (2018) Precision NanoSystems, Inc., describing methods of making lipid nanoparticles, including suitable ratios for various constituents). Briefly, appropriate amounts of the lipidoid, the sphingolipid helper lipid, the cholesterol or cholesterol derivative and PEG-based material are mixed in an appropriate solvent, such as 90% ethanol and 10% 10 mM sodium citrate and mixed with an appropriate amount of the cargo, such as siRNA in 10 mM sodium citrate at a weight ratio of siRNA or mRNA to the (lipidoid+cholesterol or cholesterol derivative+ helper lipid+PEG-based material) of, for example and without limitation of 1:2-1000, such as from 1:4 to 1:50, e.g., 1:10. For siRNA and mRNA, the lipidoid: siRNA ratio may range from 2:1 to 30:1, for example for siRNA, the lipidoid: siRNA ratio may be 5:1, and for mRNA, the lipidoid: mRNA ratio may be 10:1. The amount of helper lipid in the lipid particle may range from 10 to 80 mol % of the amounts of total lipids, e.g., lipidoid+cholesterol or cholesterol derivative+helper lipid+PEG-based material in the lipid particle. The lipid particles may be formed in an automated device (such as a microfluidic device) or by rapid pipetting. Particles may be diluted in a suitable aqueous solvent, such as PBS, and optionally dialyzed against the same or a different aqueous solvent.

If the particles prepared by any of the above methods have a size range outside of the desired range, the particles can be sized, for example, using a sieve or filter. The particle may also be coated. The particle may be coated with a targeting agent.

The lipid-containing particles comprise cholesterol or a derivative thereof, such as 3β[N—(N′,N′-dimethylaminoethane)-carbamoyl]cholesterol (DC-cholesterol). The lipid-containing particles comprise a PEG (poly(oxyethylene))-based material, such as a PEGylated fatty acid-containing compound or PEG-containing block copolymer, such as a polaxamer. Non-limiting examples of PEG-based materials include: PEG-ceramide, PEG-DMG, PEG-PE, poloxamer, or DSPE carboxy PEG. For instance, in certain embodiments, the PEG-based material is C14 PEG2000 DMG, C15 PEG2000 DMG, C16 PEG2000 DMG, C18 PEG2000 DMG, C14 PEG 2000 ceramide, C15 PEG2000 ceramide, C16 PEG2000 ceramide, C18 PEG2000 ceramide, C14 PEG2000 PE, C15 PEG2000 PE, C16 PEG2000 PE, C18 PEG2000 PE, C14 PEG350 PE, C14 PEG5000 PE, poloxamer F-127, poloxamer F-68, poloxamer L-64, or DSPE carboxy PEG.

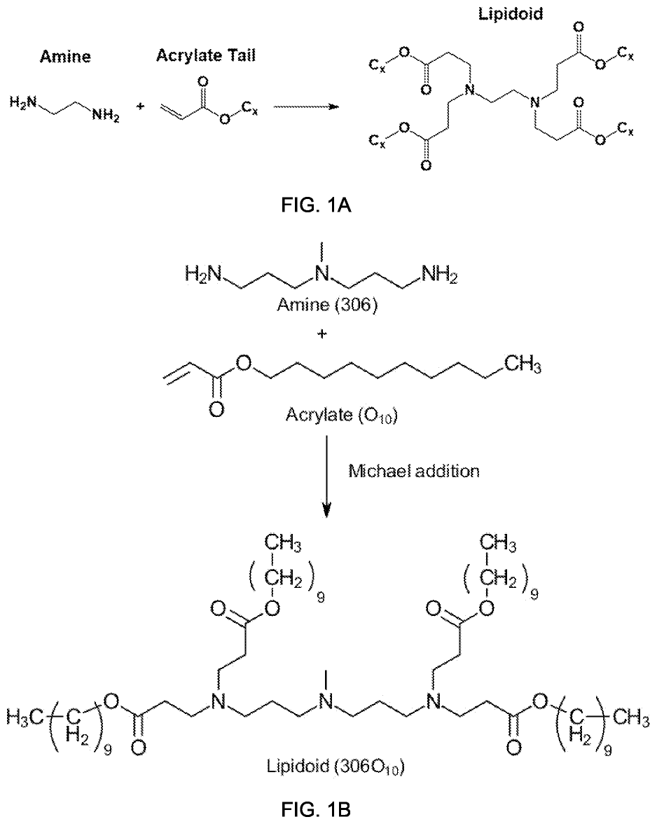

A lipidoid is a lipid-like molecule. An ionizable lipidoid is a lipidoid that forms an ion in acidic or basic conditions. Non-limiting examples of ionizable lipidoids are provided in U.S. Pat. No. 9,439,968, generally forming lipidoids by conjugate addition of alkyl-acrylates to amines. A general synthesis scheme of useful amino-lipidoids prepared from amines and alkyl-acrylates is shown in FIG. 1A. Also provided are useful amines, e.g., designated as 25, 32, 306, etc., and structures of alkyl-acrylates, e.g., O₁₀, O₁₁, O₁₂, O₁₃, and O₁₄. Lipidoids are designated in the examples below in reference to the amine and alkyl-acrylate used to make the ionizable lipidoid, e.g., 306O₁₀, referring to N¹-(3-aminopropyl)-N¹-methylpropane-1,3-diamine conjugated to decyl acrylate, as shown in FIG. 1B, with the technical name for 306O₁₀ being tetrakis(decyl) 3,3′,3″,3′″-(((methylazanediyl)bis(propane-3,1-diyl))bis(azanetriyl))tetrapropionate.

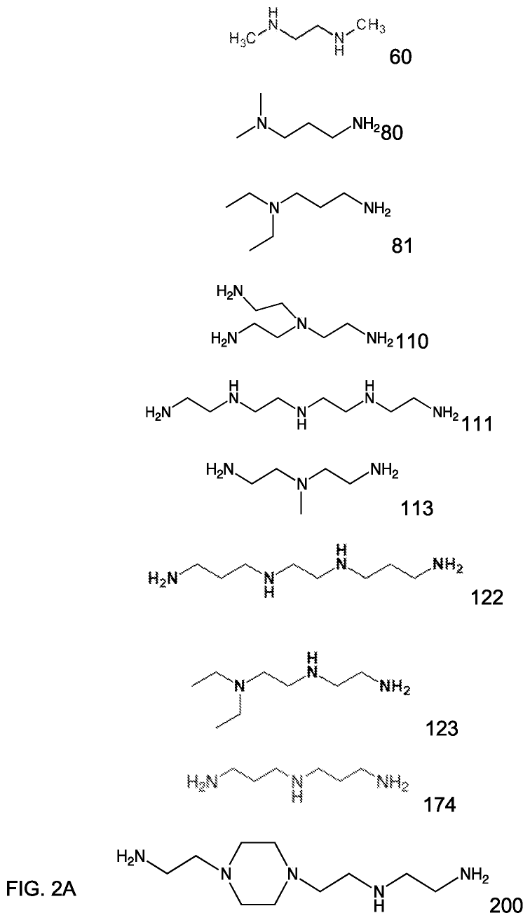

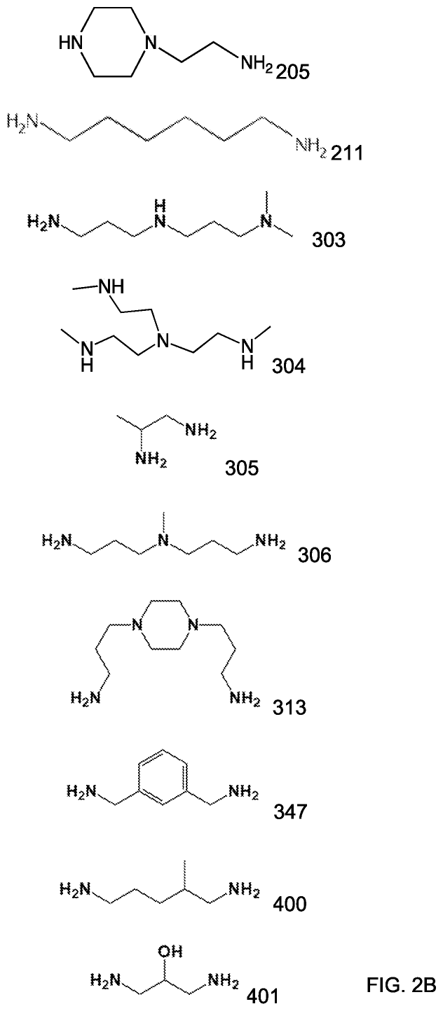

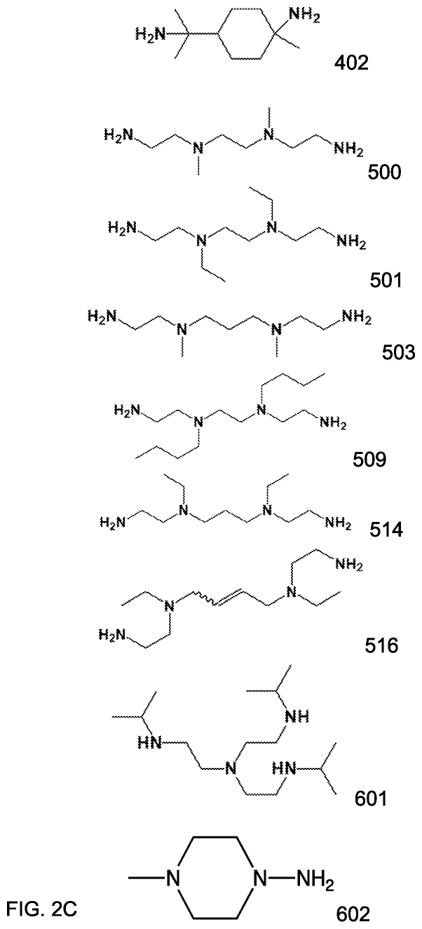

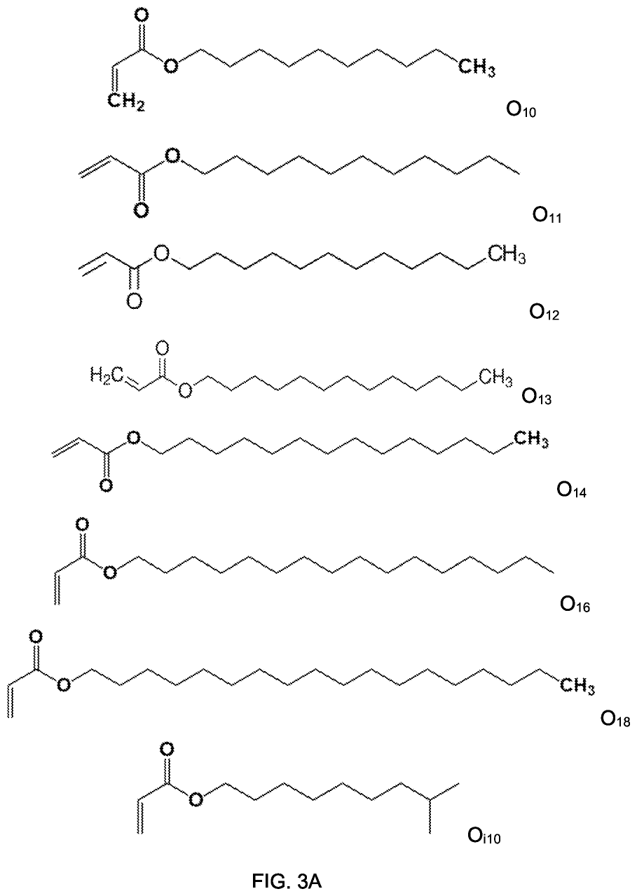

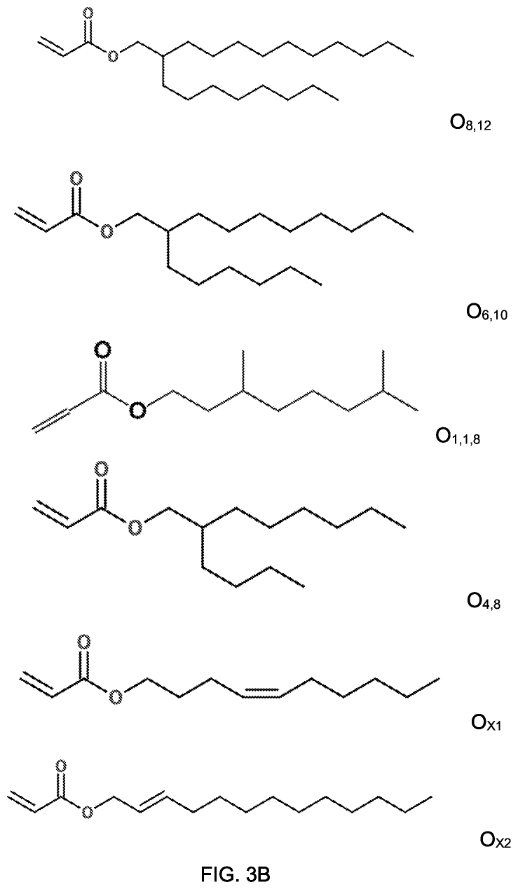

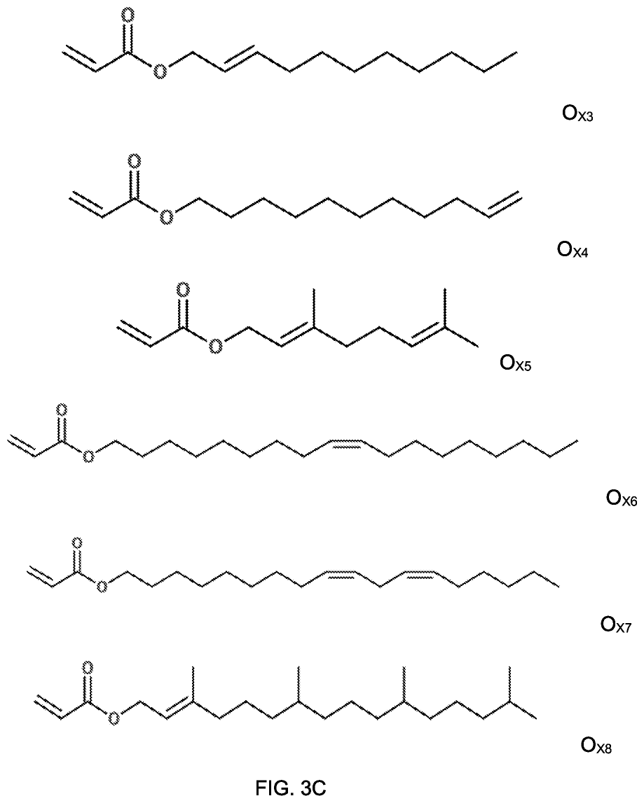

Lipidoids for preparation of LNPs for delivery to CNS tissue (including cells, tissues, organs, and/or organ systems) may include any combination, e.g., by Michael's addition, of an alkylamine having from one to five amine moieties, and an alkyl or alkenyl acrylate having a pKa in the range of from 3 to 7. Lipidoids for preparation of LNPs for delivery to the CNS may include any combinatorial permutation of an amine depicted in FIGS. 2A-2C, and one or more acrylate depicted in FIGS. 3A-3C, e.g., as depicted in FIGS. 1A and 1B. Lipidoids for preparation of lipid-containing particles for delivery to mucosa may include any combinatorial permutation of an amine depicted in FIGS. 2A-2C, and one or more acrylate depicted in FIGS. 3A-3C, having a pKa in the range of from 3 to 7, for example from 5 to 7. Additional examples of lipidoids are described in U.S. patent application Publication Nos. US20110256175A1 and US20200109113A1, and U.S. Pat. Nos. 7,939,505B2, 8,802,863B2, 8,969,353B2, 9,139,554B2, and 9,227,917B2, incorporated herein by reference for their description of additional exemplary lipidoid compounds, and uses therefor.

The lipidoid may be chosen from a lipidoid depicted in and described in the context of FIGS. 2A-3C, such as 306_{Oi10}, 306O₁₀, 503Oᵢ₁₀, and 402O_{6,10}.

Also described herein are methods and formulations, e.g., drug products that comprise lipid nanoparticles for delivery to cells, tissue, and organs of the CNS, e.g. neurons and neuronal tissue, including, without limitation: astrocytes, neurons, oligodendrocytes, neural stem cells, lymphocytes, and/or microglia cells. For use in patients, lipid nanoparticles may rely on the ionizable lipids to be neutrally charged at physiological pH (i.e., 7.4). When the lipid nanoparticles are taken up by cells, they may be trapped in endosomes which become increasingly acidic to degrade the endosome components. The ionizable lipids (lipidoids) are designed to ionize, that is, become positively charged in the acidic endosome to cause endosome membrane rupture, releasing nucleic acid cargo into the cytoplasm to allow therapeutic effect.

Intrathecal administration of therapeutic agents, e.g. APIs (Active Pharmaceutical Ingredients) such as the nucleic acid in the LNPs described herein, refers to injection into a patient's cerebrospinal fluid, e.g., into the subarachnoid space of the brain or spinal cord, e.g. to the spine, and commonly to the lumbar spine (see, e.g., Shah N, et al. Intrathecal Delivery System. [Updated 2022 Dec. 9]. In: StatPearls [Internet]. Treasure Island (FL): StatPearls Publishing; 2023 January-). Devices for intrathecal delivery include injection devices, intrathecal portals, pumps, catheters, among other turnkey or generic devices. The LNPs described herein may be provided as part of a kit, comprising a syringe for injection of the LNPs, or other reservoir or container, e.g. suitable for use in direct intrathecal injection, infusion using a pump or other device, such as, without limitation, the Intrathecal Catheter System, available from Harvard Apparatus of Holliston Massachusetts, or a pump, especially where repeated dosing is needed, such as the SynchroMed™ II Intrathecal Pump, and an associated catheter, available from Medtronic of Minneapolis, Minnesota. Brainstem injection may be similarly accomplished.

Intracerebroventricular (ICV) injection, including injection or infusion, that is, delivery of an API via the ICV route, may be performed using standard practices for such procedures and treatment. Intracerebroventricular (ICV), or intraventricular, devices have been used in the treatment of a broad range of pediatric and adult central nervous system (CNS) disorders (see, e.g., Atkinson A J Jr. Intracerebroventricular drug administration. Transl Clin Pharmacol. 2017 September; 25(3):117-124. doi: 10.12793/tcp.2017.25.3.117. Epub 2017 Sep. 15). An ICV portal, which may be referred to as an Ommaya reservoir, may be used to deliver via the ICV route (see, e.g., Zubair A, De Jesus O. Ommaya Reservoir. [Updated 2023 Feb. 12]. In: StatPearls [Internet]. Treasure Island (FL): StatPearls Publishing; 2023 January-).

The LNP compositions (e.g., API), may be compounded or formulated according to standard pharmaceutical practices, and may be administered in a suitable carrier, such as water, or water comprising suitable salts and/or buffers, as are used for intrathecal or ICV delivery routes, including, for example and without limitation, water, saline such as normal saline (e.g. 0.9% saline), buffered saline such as phosphate-buffered saline (PBS), Ringer's solution, lactated Ringer's solution, or any other suitable carrier, and including any other suitable excipient or active ingredient, preservative, rheology modifier, antibiotic, analgesic, etc.

By “expression” or “gene expression,” it is meant the overall flow of information from a gene (without limitation, a functional genetic unit for producing a gene product, such as RNA or a protein in a cell, or other expression system encoded on a nucleic acid and comprising: a transcriptional promoter and other cis-acting elements, such as response elements and/or enhancers; an expressed sequence that typically encodes a protein (open-reading frame or ORF) or functional/structural RNA, and a polyadenylation sequence), to produce a gene product (typically a protein, optionally post-translationally modified or a functional/structural RNA). By “expression of genes under transcriptional control of,” or alternately “subject to control by,” a designated sequence, it is meant gene expression from a gene containing the designated sequence operably linked (functionally attached, typically in cis) to the gene. The designated sequence may be all or part of the transcriptional elements (without limitation, promoters, enhancers and response elements), and may wholly or partially regulate and/or affect transcription of a gene. A “gene for expression of” a stated gene product is a gene capable of expressing that stated gene product when placed in a suitable environment—that is, for example, when transformed, transfected, transduced, etc. into a cell, and subjected to suitable conditions for expression. In the case of a constitutive promoter “suitable conditions” means that the gene typically need only be introduced into a host cell. In the case of an inducible promoter, “suitable conditions” means when an amount of the respective inducer is administered to the expression system (e.g., cell) effective to cause expression of the gene.

As used herein, the term “nucleic acid” refers to deoxyribonucleic acids (DNA) and ribonucleic acids (RNA). Nucleic acid analogs include, for example and without limitation: 2′-O-methyl-substituted RNA, locked nucleic acids, unlocked nucleic acids, triazole-linked DNA, peptide nucleic acids, morpholino oligomers, dideoxynucleotide oligomers, glycol nucleic acids, threose nucleic acids and combinations thereof including, optionally ribonucleotide or deoxyribonucleotide residue(s). Herein, “nucleic acid” and “oligonucleotide” which is a short, single-stranded structure made of up nucleotides, in reference to nucleic acids and nucleic acid analogs, are used interchangeably. An oligonucleotide may be referred to by the length (i.e. number of nucleotides) of the strand, through the nomenclature “-mer”. For example, an oligonucleotide of 22 nucleotides would be referred to as a 22-mer.

A “nucleic acid analog” is a composition comprising a sequence of nucleobases arranged on a substrate, such as a polymeric backbone, and can bind DNA and/or RNA by hybridization by Watson-Crick, or Watson-Crick-like hydrogen bond base pairing. Non-limiting examples of common nucleic acid analogs include peptide nucleic acids, such as yPNA, morpholino nucleic acids, phosphorothioates, locked nucleic acid (2′-O-4′-C-methylene bridge, including oxy, thio or amino versions thereof), unlocked nucleic acid (the C2′-C3′ bond is cleaved), 2′-O-methyl-substituted RNA, threose nucleic acid, glycol nucleic acid, etc.

Gene editing in any form may be used to introduce or correct polymorphisms in a tissue of the central nervous system, administerd according to methods described herein, e.g., intrathecally, intracerebrally, or to the brainstem. A single-nucleotide polymorphism or single-nucleotide variant (SNP or SNV, respectively), or other disease-associated mutation, polymorphism, variant, etc. may be detected by sequencing, PCR, RT-PCR, or any useful method, and may be corrected using one or more editing tools, such as the CRISPR and Cas9-based tools described herein. Likewise, a dominant, ancestral, wild-type, primary, normal (etc.) allele may be changed to alter a response, interaction, regulatory pathway, etc. to treat a disease, or otherwise to produce a desirable genotype or phenotype, for example in animal breeding, to treat an over-reactive response, or to treat hyperplasia or cancers. As such, whatever the reason and associated genomic sequence, whether normal or abnormal, or the degree of association of an allele with disease risk, gene editing may be used to alter a relevant nucleotide sequence in a desired manner.

A CRISPR-CAS9 editing (Clustered Regularly Interspaced Short Palindromic Repeats (CRISPR)-CRISPR-associated protein 9 (Cas9)) system, single base editing, or prime editing, as examples of gene editing methods, may be used to remove or edit one or more nucleobases, e.g., to substitute, insert, and/or delete sequences present in the genome of a subject or patient. CRISPR-Cas9 may be used to inactivate or correct a gene, or base editors or prime editors may be utilized. The CRISPR-Cas9 system, as well as single base editors, include a guide RNA (gRNA) or single guide RNA (sgRNA) and a CRISPR-associated protein 9 (Cas9) nuclease. Identification of the DNA target strand, and methods of implementing a change in the target DNA (e.g., gene knock out in the target DNA strand, knock-in of a desired sequence, or base substitutions) are within the abilities of one of ordinary skill in the art.

The non-target DNA strand includes a specific protospacer adjacent motif (PAM) in order for the gRNA to bind to the target DNA strand. The PAM is a short nucleotide motif that is found 3′ to the target site. For the CRISPR-Cas9 system, the PAM may be 5′-NGG-3′, where N is any nucleotide and G is guanine. The Cas9 nuclease cuts 3 to 4 nucleotides upstream of the PAM sequence. The locations in the genome that can be targeted by different Cas proteins may be limited by the locations of the PAM sequences and are known to those of ordinary skill in the art.

In Crispr-Cas9 editing, when the Cas9 nuclease binds with the PAM and the gRNA binds with the target DNA strand, a double-strand break is caused in the gRNA sequence. Endogenous repair mechanisms, such as non-homologous end joining (NHEJ), microhomology-mediated end joining (MMEJ), or homologous directed repair (HDR), are triggered by the double-strand break and result in a gene knock out in the target DNA strand or a knock-in of a desired sequence if a DNA template is present. The DNA template includes the desired sequence, which is flanked by sequences that are homologous to the region upstream and downstream of the double-stranded break.

The gRNA includes a CRISPR RNA (crRNA), which is a 17-20 nucleotide sequence that is complementary to the target DNA strand, and a tracrRNA, which serves as a binding scaffold for the Cas9 nuclease. The crRNA and the tracrRNA may exist as two separate RNA molecules. Alternatively, the sgRNA may comprise both the crRNA sequence and the tracrRNA sequence, where the crRNA sequence is fused to the scaffold tracrRNA sequence. gRNAs of base editing methods as described below, have canonical structures specific to each technique. One of ordinary skill in the art would select a gRNA or sgRNA that maximizes the on-target DNA cleavage efficiency, while also minimizing unintentional off-target binding and cleavage effects (see, Konstantakos et al. “CRISPR-Cas9 gRNA efficiency prediction: an overview of predictive tools and the role of deep learning. Nucleic Acids Res., 2022, 50(7):3616-3637 and “The Complete Guide to Understanding CRISPR sgRNA”, Synthego, 2023, www.synthego.com/guide/how-to-use-crispr/sgrna).

Alternatively, a base editing system may be used to convert a G to another nucleobase. Base editing is a genome-editing technique that uses DNA base editors to directly generate precise point mutations without generating a double-strand break without double-strand breaks. The DNA base editors may comprise fusions between a catalytically dead Cas9 (dCas9) or a nickase Cas9 (nCas9) fused to a single-stranded DNA (ssDNA)-specific deaminase and a single guide RNA (sgRNA). The d/nCas9 recognizes a specific sequence named protospacer adjacent motif (PAM) and the DNA unwinds thanks to the complementarity between the sgRNA and the DNA sequence usually located upstream of the PAM (“protospacer”). Then, the opposite DNA strand is accessible to the deaminase that converts the bases located in a specific DNA stretch of the protospacer (see, e.g., Antoniou P, et al. Base and Prime Editing Technologies for Blood Disorders. Front Genome Ed. 2021 Jan. 28; 3:618406). Upon binding of the DNA base editor to the target DNA strand, base pairing between the sgRNA and the target DNA strand results in the displacement of a small segment of ssDNA as an “R-loop”. The DNA bases within the ssDNA are therefore substrates for deamination and are subsequently modified by the deaminase enzyme. The DNA base editor may be a cytosine base editor (CBE), which converts a C/G base pair into a T/A base pair or an adenine base editor (ABE) which converts an A/T base pair into a G/C base pair (see, e.g., Qi et al. “Base Editing Mediated Generation of Point Mutations Into Human Pluripotent Stem Cells for Modeling Disease”, Frontiers in Cell and Developmental Biology, 2020, 8 (590581):1-12; Nishida K, et al. Targeted nucleotide editing using hybrid prokaryotic and vertebrate adaptive immune systems. Science. 2016 Sep. 16; 353 (6305): aaf8729; Komor A C, et al. Programmable editing of a target base in genomic DNA without double-stranded DNA cleavage. Nature. 2016 May 19; 533 (7603):420-4; and Gaudelli N M, et al. Programmable base editing of A•T to G•C in genomic DNA without DNA cleavage. Nature. 2017 Nov. 23; 551(7681):464-471).

Another method of editing SNV is prime editing, which is disclosed in U.S. Pat. No. 11,447,770 B1, incorporated herein by reference for its technical disclosure, and related publications (see also, International Patent Publication No. WO2020191242 A1 and Anzalone A V, et al. Search-and-replace genome editing without double-strand breaks or donor DNA. Nature. 2019 December; 576(7785):149-157). Prime editors (PEs), including a complete description of pegRNA are provided in those references, as well as methods of in vivo delivery of prime editor materials, such as viral vectors, e.g., AAV particles encoding prime editors, are described in that patent publication and related publications.

Prime editing is a “search and replace” gene editing method in which Moloney Murine Leukemia Virus Reverse Transcriptase (M-MLV RT) is fused to the C-terminus of Cas9 H840A nickase. The fusion enzyme is installs targeted insertions, deletions, and all possible base-to-base conversions using a prime editing guide RNA (pegRNA). The pegRNA directs the nickase to the target site by homology to a genomic DNA locus. The longer pegRNA also encodes a primer binding site (PBS) and the desired edits on an RT template. Prime editing has gone through a number of versions. In PE1, the pegRNA directs the Cas9 nickase to the target sequence where it nicks the non-target strand and generates a 3′ flap. The 3′ flap binds to the primer binding site (PBS) of the pegRNA and the desired edit is incorporated into the DNA by reverse transcription. The edited DNA strand displaces the unedited 5′ flap and the resulting heteroduplex is resolved by the cell's mismatch repair (MMR) system. Alternatively, the edited 3′ flap may be excised and the target sequence will remain unchanged but available as a substrate for another round of prime editing.

In the PE2 system, mutations were introduced into the RT enzyme to increase activity, enhance binding between the template and PBS, increase processivity, and improve thermostability. PE3 uses the PE2 Cas9 nickase-pentamutant RT fusion enzyme and pegRNA plus an additional simple sgRNA, which directs the Cas9 nickase to nick the unedited strand at a nearby site. The newly edited strand is then favored as the template for repair during heteroduplex resolution. The process of double nicking, however, increases indel formation slightly. Designing the sgRNA with a spacer that only binds the edited strand, as in the PE3b system, guides nicking of the unedited strand only after the edit has occurred. PE4 and PE5 also have been described (Chen P J, et al. Enhanced prime editing systems by manipulating cellular determinants of editing outcomes. Cell. 2021 Oct. 28; 184 (22):5635-5652.e29). Plasmids useful for performing prime editing are commercially-available from Addgene (www.addgene.org/crispr/prime-edit/). “Prime editing” includes all variations of prime editing methods, including, without limitation, PE1, PE2, PE3, PE3b, PE4, and PE5 versions. pegRNA includes variations thereof for use in the many variations of prime editing, such as, without limitation, epegRNA (Nelson J W, et al. Engineered pegRNAs improve prime editing efficiency. Nat Biotechnol. 2022 March; 40(3):402-410).

Computer-based tools have been developed for automated generation of pegRNA (see, e.g., Morris et al. Automated design of CRISPR prime editors for 56,000 human pathogenic variants. iScience. 2021 Oct. 30; 24(11):103380, and the tool, Prime Editing Design Tool, for identification of useful pegRNAs is provided at primeedit.nygenome.org/; Hwang G H, et al. PE-Designer and PE-Analyzer: web-based design and analysis tools for CRISPR prime editing. Nucleic Acids Res. 2021 Jul. 2; 49(W1): W499-W504; Hsu J Y, et al. PrimeDesign software for rapid and simplified design of prime editing guide RNAs. Nat Commun. 2021 Feb. 15; 12(1):1034; and Chow R D, et al. A web tool for the design of prime-editing guide RNAs. Nat Biomed Eng. 2021 February; 5(2):190-194). pegRNAs may contain a protospacer sequence for recognizing the target sequence, a reverse transcriptase template (RTT) that contains the desired edit, and a primer binding site (PBS) for the activation of reverse transcriptase. As above, several types of PEs have been developed and different gRNAs can be used, depending on the type of prime editor (e.g. PE2 vs PE3). For example, in contrast with PE2, which requires only a pegRNA, PE3 also requires a nicking guide RNA (ngRNA) to increase the prime editing efficiency (Hwang G H, et al. Nucleic Acids Res. 2021 Jul. 2; 49(W1):W499-W504). Although pegRNAs and ngRNAs, when applied, can be developed by a person of ordinary skill without use of a computer, that person may employ computational tools, such as those described above, to effectively design a pegRNA, and other useful reagents, for prime editing.

The CRISPR/Cas9, base editing, and prime editing, necessary components, e.g., gRNA, template, pegRNA, ngRNA, nucleic acid (RNA, DNA, or a mixture thereof, including nucleic acid analogs) encoding Cas9, Cas9 nickase, or Cas9 fusion proteins, etc. may be delivered by any effective means, but in the context of the present disclosure, by LNP. The transferred material may take any useful form, but may include a DNA plasmid or recombinant viral genome containing sequences for expression of necessary reagents; and/or mRNA for translation of the reagents, along with suitable guide RNA, e.g. gRNA or pegRNA, and other useful nucleic acid reagents, such as ngRNA. DNA or RNA useful for implementing a gene editing method, e.g. a CRISPR/Cas9 editing method, including single base editing, such as CBEs ABEs or prime editing, may be delivered to the CNS consistent with and/or according to methods and reagents described herein.

Gene editing tools have been delivered by LNP, but the present disclosure provides enhanced delivery to the CNS through use of sphingolipid-modified LNPs. In one example, Rosenblum et al. (Rosenblum D, et al. CRISPR-Cas9 genome editing using targeted lipid nanoparticles for cancer therapy. Sci Adv. 2020 Nov. 18; 6(47):eabc9450) delivered gene editing tools intracerebrally against PLK1, to treat aggressive orthotopic glioblastoma, with good results. Rosenblum et al. also describe antibody targeting of the described LNPs. See, also, Onuma H, et al. (Onuma H, et al. Lipid nanoparticle-based ribonucleoprotein delivery for in vivo genome editing. J Control Release. 2023 March; 355:406-416) and Li B, et al. (Li B, et al. Combinatorial design of nanoparticles for pulmonary mRNA delivery and genome editing. Nat Biotechnol. 2023 Mar. 30), among others. As such, LNPs as described herein are expected to be able to deliver therapeutic gene editing nucleic acids for effective gene editing.

LNPs have been used to deliver DNA. LNPs are commonly thought of as RNA delivery compositions. That said, DNA can effectively be delivered, such as, without limitation, plasmid DNA, linear DNA, and viral genomes such as adenovirus (Ad) or adeno-associated virus (AAV) genomes. The LNPs described herein can be used to deliver DNA. The ratios of ingredients of the sphingolipid-containing LNPs may need to be manipulated to optimize delivery, but this is well within the abilities of a person of ordinary skill. See, e.g., Zhu Y et al. (Multi-step screening of DNA/lipid nanoparticles and co-delivery with siRNA to enhance and prolong gene expression. Nat Commun. 2022 Jul. 25; 13(1):4282), Algarni et al. (In vivo delivery of plasmid DNA by lipid nanoparticles: the influence of ionizable cationic lipids on organ-selective gene expression. Biomater Sci. 2022 May 31; 10(11):2940-2952), and Scalzo et al. (Ionizable Lipid Nanoparticle-Mediated Delivery of Plasmid DNA in Cardiomyocytes. Int J Nanomedicine. 2022 Jun. 30; 17:2865-2881), among others. As such, LNPs as described herein are expected to be able to deliver therapeutic DNA for effective production of gene products.

Messenger RNA has been successfully delivered to the brain. For example, ischemic stroke has been shown to be treatable with delivery of heme oxygenase-1 (HO1) mRNA via self-replicating mRNA (Rep-mRNA) developed using a replicon system from Venezuelan Equine Encephalitis virus (Kim M, et al. Delivery of self-replicating messenger RNA into the brain for the treatment of ischemic stroke. J Control Release. 2022 October; 350:471-485, see also, NCBI Reference Sequence: NM_002133.3 for an example of a sequence of a deliverable HO1 mRNA). Intracrainial delivery of synthetic modified tumor necrosis factor-related apoptosis-inducing ligand (TRAIL) mRNA, using TransIT®-mRNA and jetPEI®, showed efficacy in a murine glioblastoma model (Peng H, et al. Intracranial delivery of synthetic mRNA to suppress glioblastoma. Mol Ther Oncolytics. 2021 Dec. 14; 24:160-170). Lastly, intraventricular delivery of mRNA encoding brain-derived neurotrophic factor (BDNF) using a polymer-based carrier, polyplex nanomicelle, was shown to increase the survival rate of hippocampal neurons after transient global ischemia (Fukushima Y, et al. Treatment of ischemic neuronal death by introducing brain-derived neurotrophic factor mRNA using polyplex nanomicelle. Biomaterials. 2021 March; 270:120681). TRAIL and BDNF mRNA sequences are widely known and are available. Production of mRNA for use in the sphingolipid LNPs described herein may be accomplished using any suitable gene expression system and isolating the mRNA according to well-established methods (see, e.g., Rosa S S, et al. mRNA vaccines manufacturing: Challenges and bottlenecks. Vaccine. 2021 Apr. 15; 39 (16):2190-2200). A number of companies, such as ThermoFisher Scientific, among others, provide bulk mRNA production services. As such, LNPs as described herein are expected to be able to deliver therapeutic RNAs for effective treatment of diseases such as ischemia, stroke, ischemia/reperfusion injury, neuroinflammation, neurodegenerative diseases, monogenic neurological disorders, and cancers, such as, without limitation, glioblastoma.

### Example 1—Helper Lipid Screening

Lipid nanoparticles were formulated with different helper lipids for mRNA delivery to murine, and human microglial cells.

### Materials and Methods

The mRNA was N(1)-methylpseudouridine (m1ψ)-modified mRNA encoding green fluorescent protein (GFP mRNA).

The lipid nanoparticles (LNPs) were formulated with 16 mol % helper lipid, 35 mol % ionizable lipidoid (306_{Oi10} as a proof-of concept example), 46.5 mol % cholesterol and 2.5 mol % 1,2-dimyristoyl-sn-glycero-3-phosphoethanolamine-N-[methoxy (polyethylene glycol)-2000] (ammonium salt) (C14-PEG2000). The helper lipid was either 1,2-dioleoyl-sn-glycero-3-phosphoethanolamine (DOPE), 1,2-dioleoyl-sn-glycero-3-phospho-L-serine (sodium salt) (DOPS), 1,2-dioleoyl-sn-glycero-3-phosphocholine (DOPC), 1,2-dioleoyl-sn-glycero-3-phospho-(1′-rac-glycerol) (sodium salt) (PG), 1,2-dioleoyl-sn-glycero-3-phosphate (sodium salt) (PA), 1,2-dioleoyl-3-trimethylammonium-propane (chloride salt) (DOTAP), glycero-ethylphosphocholine EPC, brain sphingomyelin (Brain SM), brain ganglioside extract, brain sulfatide, N-oleoyl-D-erythro-sphingosine (referred to in the examples as Ceramide), C18 glucosyl glycosphingolipid (referred to in the examples as glucosyl ceramide), C18 lactosyl glycosphingolipid (referred to in the examples as lactosyl ceramide), or C18 galactosyl glycosphingolipid (referred to in the examples as galactosyl ceramide).

The lipidoid, helper lipid, cholesterol, and C14-PEG2000 were dissolved in reagent grade ethanol at 1-10 milligrams per milliliter (mg/mL). GFP mRNA was dissolved in 10 mM sodium citrate monobasic. Citrate buffer was added to the lipid solutions at 1:10 volume ratio. The resultant lipid solution was added to an equal volume of mRNA solution at a 10:1 lipidoid: mRNA mass ratio and then mixed thoroughly. Finally, an equal volume of phosphate-buffered saline (PBS) was added to the ethanol-citrate mixture, and this was mixed thoroughly. Lipid nanoparticles for in vitro and in vivo studies were formulated at final mRNA concentrations of 5 g/mL and 90 μg/mL, respectively. Lipid nanoparticles used for in vivo studies were dialyzed against 2 liters (L) of PBS in 3 kiloDalton (kDa) molecular weight cut off dialysis cassettes for 90 minutes.

### Cell Culture—Human Microglia Cells (HCM3)

Human microglia cells (HCM3) were cultured in EMEM/10% FBS/1% Pen/Strep/0.1% Fungizone at 37° C./5% CO₂ and split with trypsin prior to confluency. Cells were allowed to adhere or settle for 24 hours prior to LNP administration. Twenty microliters of LNPs per well at an original concentration of 5 μg/mL GFP mRNA were added into cell culture media then allowed to incubate for 110 hours. HCM3 control cells were treated with PBS or naked GFP mRNA. Transfection efficiency was measured in real time every two hours using an IncuCyte ZOOM Live-Cell imaging system.

### Cell Culture—Mouse Microglia Cells (SIM-A9)

Mouse microglia SIM-A9 cells were cultured in DMEM-F12/10% FBS/5% Horse serum/1% Pen/Strep/0.1% Fungizone at 37° C./5% CO₂ and split with trypsin prior to confluency. Cells were allowed to adhere or settle for 24 hours prior to LNP administration. A physiologically relevant mucin layer was created by adding 25 μL of 5% mucin diluted in media covered by an additional 155 μL of media. Twenty microliters of LNPs per well at an original concentration of 5 μg/mL GFP mRNA were added into cell culture media then allowed to incubate for 110 hours. SIM-A9 control cells were treated with PBS or naked GFP mRNA. Transfection efficiency was measured in real time every two hours using an IncuCyte ZOOM Live-Cell imaging system.

### Cell Culture—Human Glioblastoma Cells (U87MG)

Human glioblastoma U87MG cells were cultured in DMEM/10% FBS/1% Pen/Strep/0.1% Fungizone at 37° C./5% CO₂ and split with trypsin prior to confluency. Cells were allowed to adhere or settle for 24 hours prior to LNP administration. A physiologically relevant mucin layer was created by adding 25 μL of 5% mucin diluted in media covered by an additional 155 μL of media. Twenty microliters of LNPs per well at an original concentration of 5 μg/mL GFP mRNA were added into cell culture media then allowed to incubate for 110 hours. U87MG control cells were treated with PBS or naked GFP mRNA. Transfection efficiency was measured in real time every two hours using an IncuCyte ZOOM Live-Cell imaging system.

### Cell Culture—Mouse Glioblastoma Cells (GL261)

Mouse glioblastoma GL261 cells were cultured in DMEM/10% FBS/1% Pen/Strep/0.1% Fungizone at 37° C./5% CO₂ and split with trypsin prior to confluency. Cells were allowed to adhere or settle for 24 hours prior to LNP administration. A physiologically relevant mucin layer was created by adding 25 μL of 5% mucin diluted in media covered by an additional 155 μL of media. Twenty microliters of LNPs per well at an original concentration of 5 g/mL GFP mRNA were added into cell culture media then allowed to incubate for 110 hours. GL261 control cells were treated with PBS or naked GFP mRNA. Transfection efficiency was measured in real time every two hours using an IncuCyte ZOOM Live-Cell imaging system.

### Results and Discussion

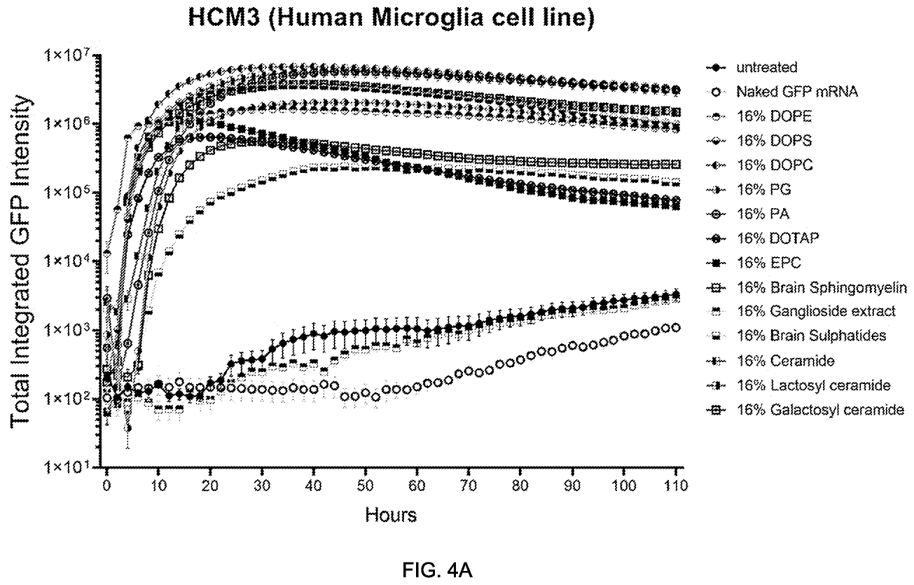

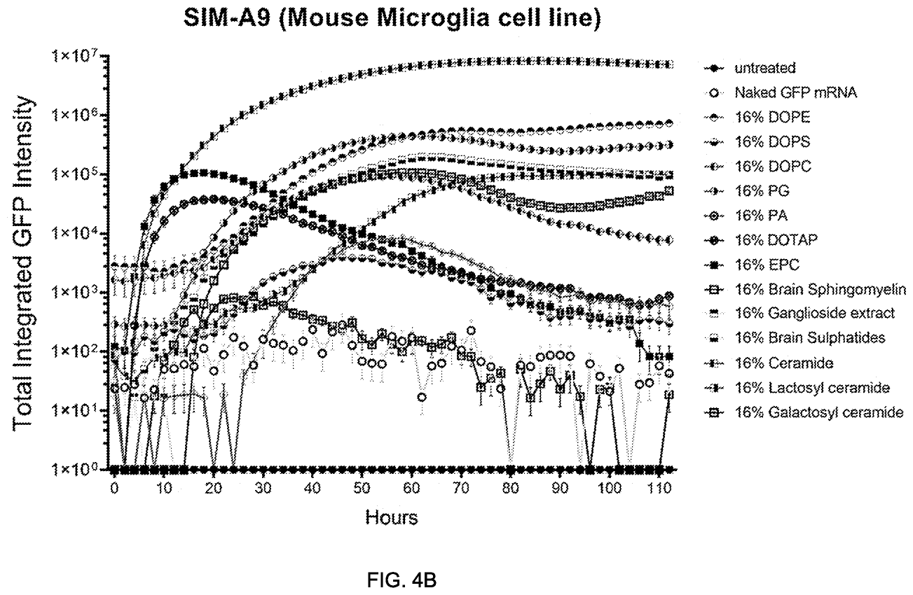

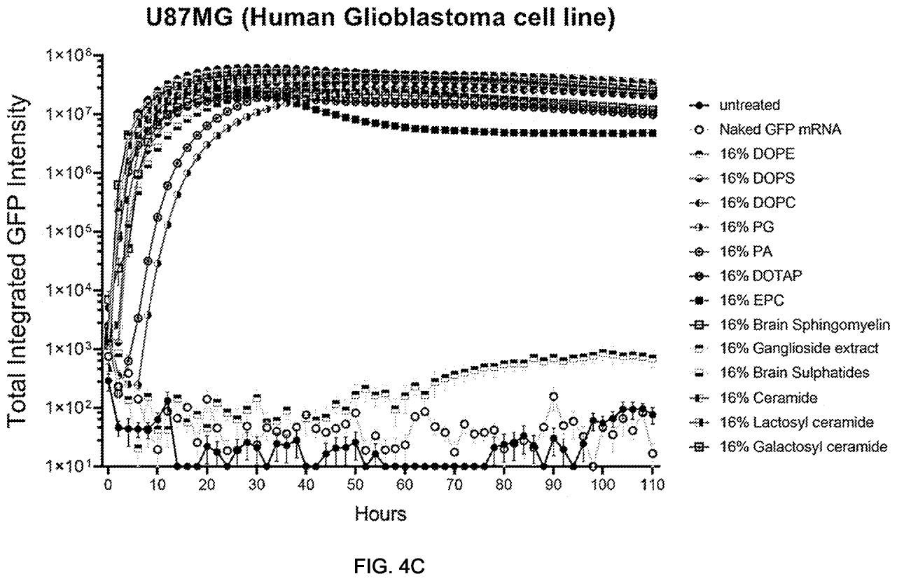

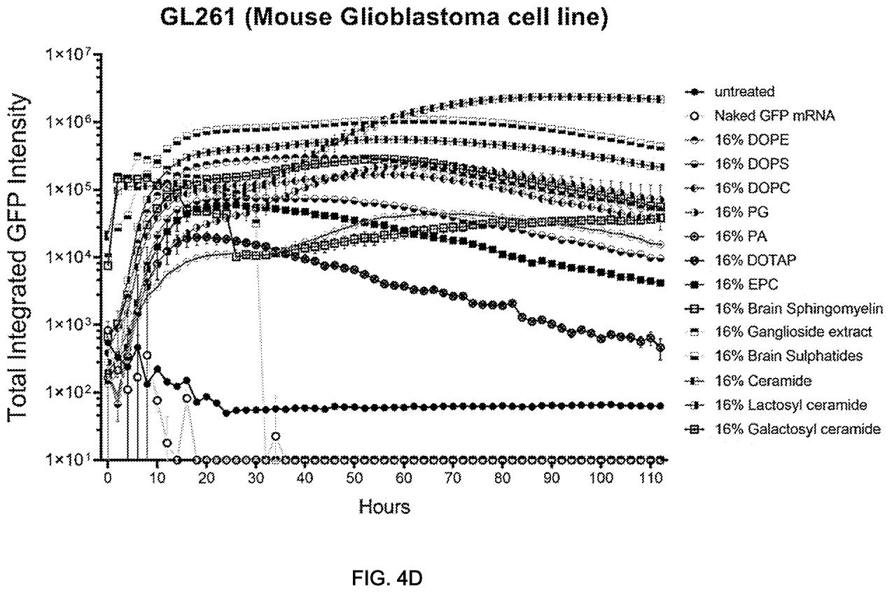

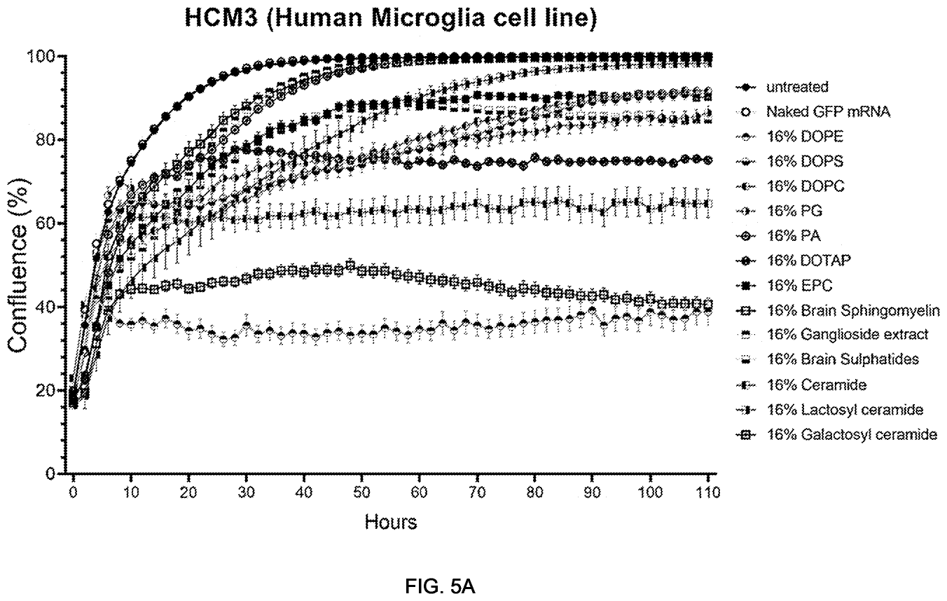

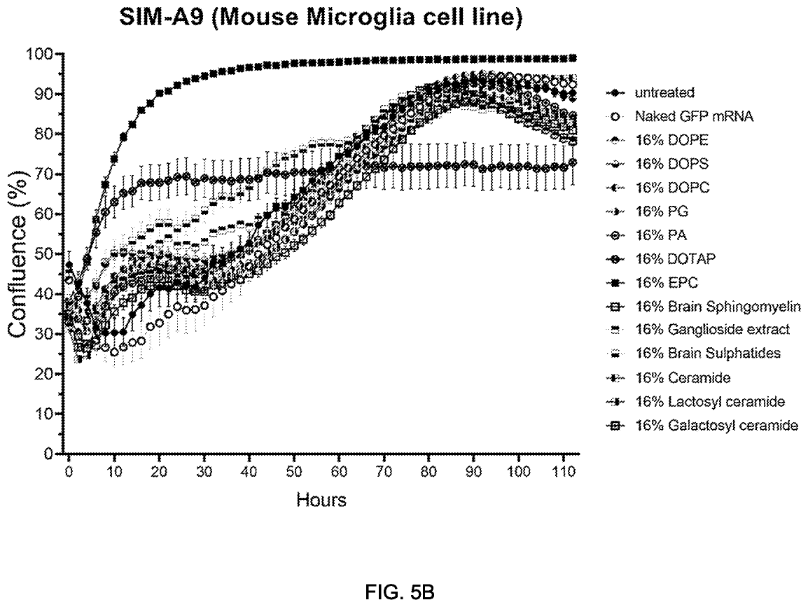

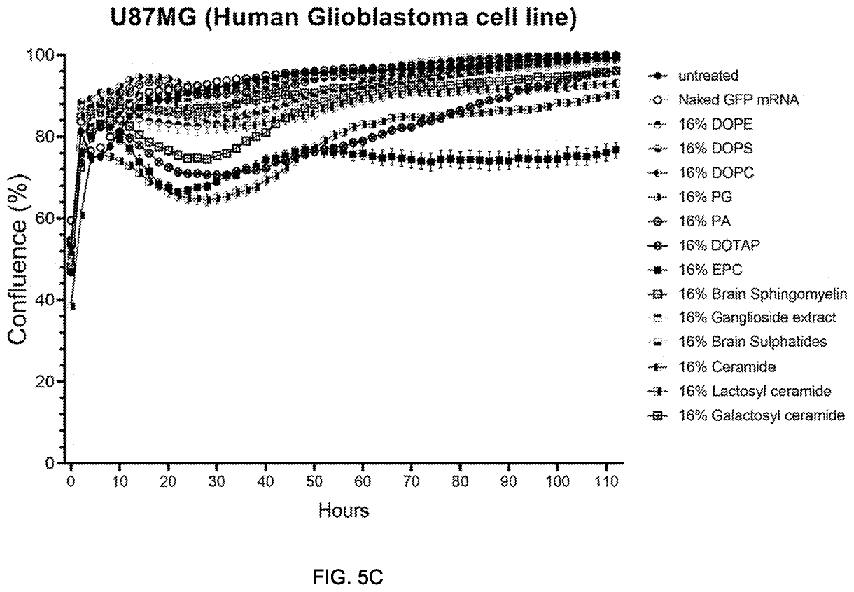

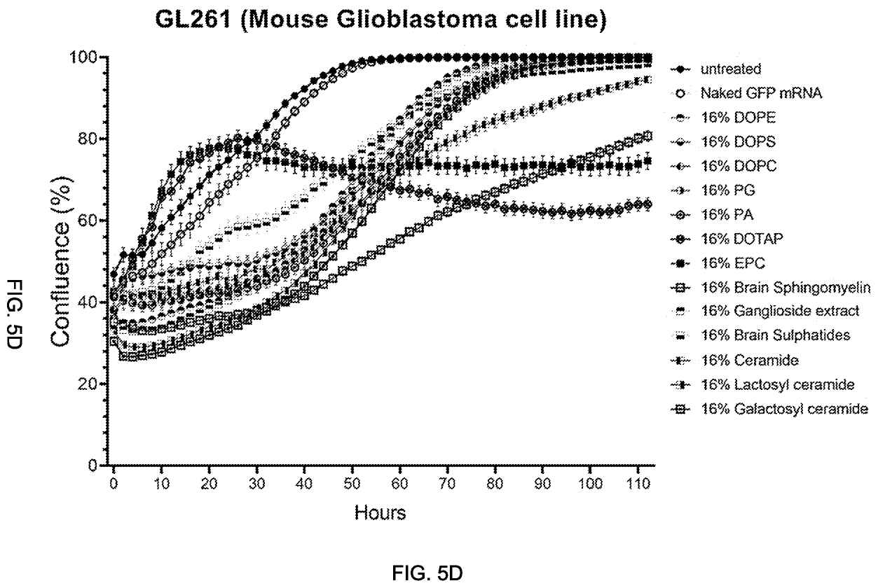

The total integrated green GFP fluorescence intensity of the HCM3, SIM-A9, U87MG, and GL261cells upon exposure to the 13 different GFP mRNA-LNPs (n=4) is shown in FIGS. 4A-4D. The cell confluence (%) of the HCM3, SIM-A9, U87MG, and GL261 cells upon exposure to the 13 different GFP mRNA-LNPs (n=4) are shown in FIGS. 5A-5D.

The lipid nanoparticles formulated with a sphingolipid helper lipid (e.g., brain sphingomyelin, brain ganglioside exact, brain sulfatide, Ceramide, lactosyl ceramide, and galactosyl ceramide) gave high transfection efficacy as compared to LNP formulations prepared using the other helper lipids.

### Example 2—In Vivo Intrathecal LNP Administration

### Materials and Methods

The LNPs were formulated with 16 mol % DOPE, 35 mol % ionizable lipidoid (306_{Oi10} as a proof-of concept example), 46.5 mol % cholesterol and 2.5 mol % C14-PEG₂₀₀₀. The mRNA was luciferase mRNA (mLuc) or a combination of mLuc and mCherry mRNA (mCherry). The LNPs were prepared according to the method described in Example 1. The LNPs were formulated to contain 0.5 mg/kg mRNA.

Male C57BL/6NCrl (Charles River) mice of at least 6 weeks of age were used for all in vivo experiments. Mice were independently injected intrathecally with (1) LNPs containing mLuc or (2) LNPs containing a combination of mLuc and mCherry. After 1 hour, 3 hours, 6 hours, 12 hours, 18 hours, 24 hours, or 48 hours post-injection, the mice that were injected with LNPs containing mLuc were injected intraperitoneally with 130 μL of 30 mg/mL D-luciferin. After 6 hours post-injection, the mice that were injected with LNPs containing mLuc and mCherry were injected intraperitoneally with 130 μL of 30 mg/mL D-luciferin. Fifteen minutes following luciferin administration, mice were euthanized via CO₂ asphyxiation and secondary cervical dislocation. Organs were removed, excess blood was blotted off, and organs were placed on black construction paper. Luminescent signal was measured using an In Vivo Imaging System (Perkin Elmer), and luminescent images were juxtaposed with brightfield images. Total luminescent flux (p/s) was calculated for each organ using Living Image@ software. Organs used for histology were immediately placed in 10% neutral buffered formalin and stored at 4° C. for 4 days before washing in PBS and storing in 70% ethanol at 4° C.

### Results and Discussion

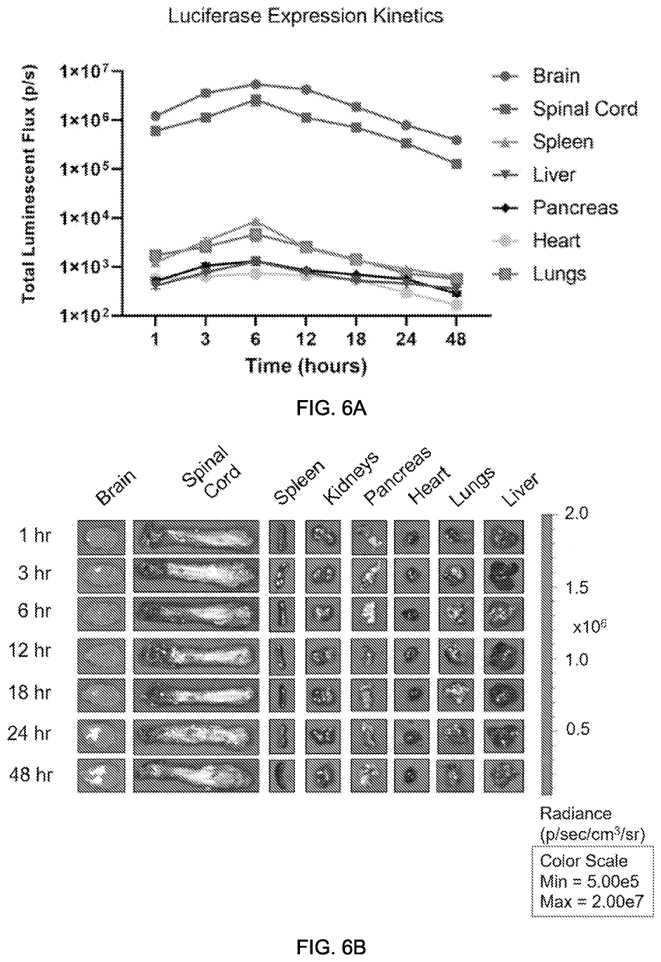

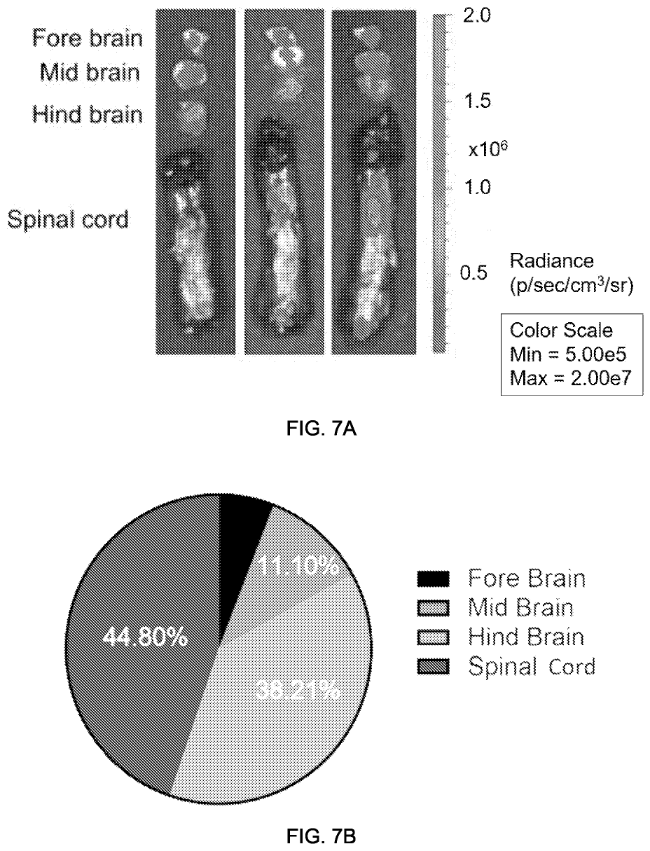

The in vivo luciferase expression kinetic results are shown in FIGS. 6A and 6B. It was determined that 3 or 6 hours was the optimal time to assess luciferase expression in mouse brain. To assess global mRNA transfection, the brain was sectioned into three regions (fore/mid/hind brain and spinal cord), which confirmed that intrathecal route enabled delivery throughout the mouse brain (see FIGS. 7A-7B).

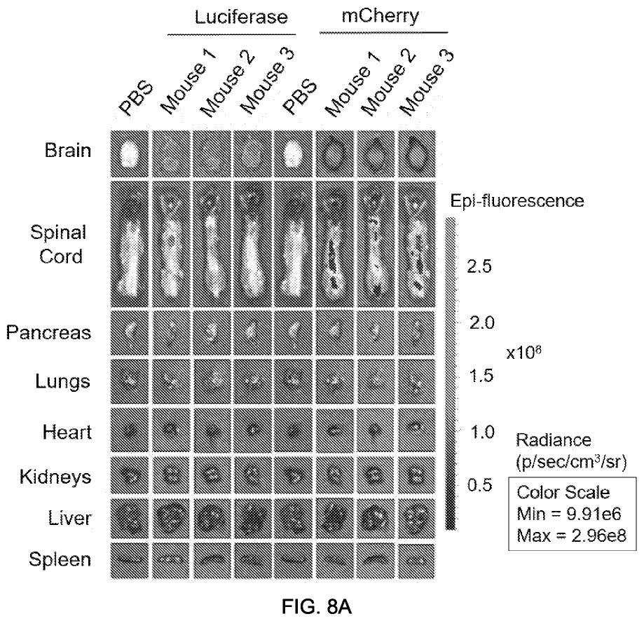

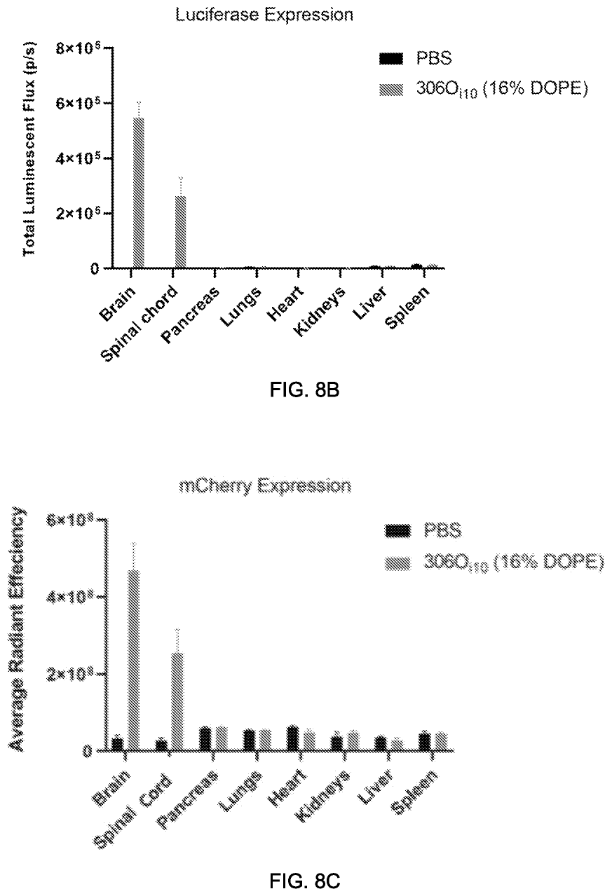

Multiplex mRNA delivery of the LNPs containing mLuc and mCherry was analyzed at 6 hours post injection. The resulting data is shown in FIGS. 8A-8C, which shows successful transfection with mRNAs for luciferase and mCherry. Thus, multiple mRNAs were delivered to the brain via LNP intrathecal injection.

### Example 3—Helper Lipid Evaluation Via In Vivo Intrathecal LNP Administration

### Materials and Methods

LNPs were independently formulated with (1) 16 mol % DOPE, 35 mol % ionizable lipidoid (306_{Oi10} as a proof-of concept example), 46.5 mol % cholesterol and 2.5 mol % C₁₄-PEG₂₀₀₀; (2) 16 mol % DOPS, 35 mol % ionizable lipidoid (306_{Oi10} as a proof-of concept example), 46.5 mol % cholesterol and 2.5 mol % C14-PEG2000; or (3) 16 mol % DOTAP, 35 mol % ionizable lipidoid (306_{Oi10} as a proof-of concept example), 46.5 mol % cholesterol and 2.5 mol % C₁₄-PEG₂₀₀₀. The mRNA was mLuc, Cre recombinase mRNA (mCre), or a combination of mRNA for Cas9 and sgRNA for murine CD81 (Synthego Corporation). A scramble guide control was obtained from Synthego Corporation. The LNPs were prepared according to the method described in Example 1. The LNPs containing mLuc or mCre were formulated to contain 0.5 mg/kg mRNA. The LNPs containing a combination of mRNA for Cas9 and sgRNA for murine CD81 were formulated to contain 0.5 mg/kg mRNA for Cas9 and 0.05 mg/kg sgRNA for murine CD81, where ratio of cas9 mRNA to sgRNA was 4.7:1 (w/w). The formulation including the scramble guide control was the same as the CD81 on-target formulation, except the guide was the scramble.

Evaluation of mRNA Delivery to the Brain

Male C57BL/6J (Charles River) mice or Male Ai9 reporter mice (Jackson Laboratory) mice of at least 6 weeks of age were used for in vivo experiments. C57BL/6NCrl mice were independently injected intrathecally with (1) DOPE helper lipid LNPs containing mLuc; (2) DOPS helper lipid LNPs containing mLuc; or (3) DOTAP helper lipid LNPs containing mLuc. Ai9 mice were independently injected intrathecally with (1) DOPE helper lipid LNPs containing mCre; (2) DOPS helper lipid LNPs containing mCre; or (4) DOTAP helper lipid LNPs containing mCre. After 6 hours post-injection, the C57BL/6NCrl mice were injected intraperitoneally with 130 μL of 30 mg/mL D-luciferin. After 96 hours post-injection, the Ai9 mice were injected intraperitoneally with 130 L of 30 mg/mL D-luciferin. The mice were euthanized and the organs were prepared and analyzed as described in Example 2.

### Cas9-Mediated Gene Knockout in Mice

Male C57BL/6 mice were independently injected intrathecally with (1) DOPE helper lipid LNPs containing mRNA for Cas9 and sgRNA for murine CD81; (2) DOPS helper lipid LNPs containing mRNA for Cas9 and sgRNA for murine CD81; or (3) DOTAP helper lipid LNPs containing mRNA for Cas9 and sgRNA for murine CD81. After one week post-injection, the C57BL/6NCrl mice were injected intraperitoneally with 130 μL of 30 mg/mL D-luciferin. The mice were euthanized and the organs were prepared and analyzed as described in Example 2. The brains from the mice were harvested, mechanically digested into single cell suspensions and analyzed using flow cytometry to assess for CD81 knockout. To assess successful expression of Cas9, a separate set of animals were euthanized at 24 hours post LNP administration.

### Results and Discussion

mRNA Delivery to the Brain

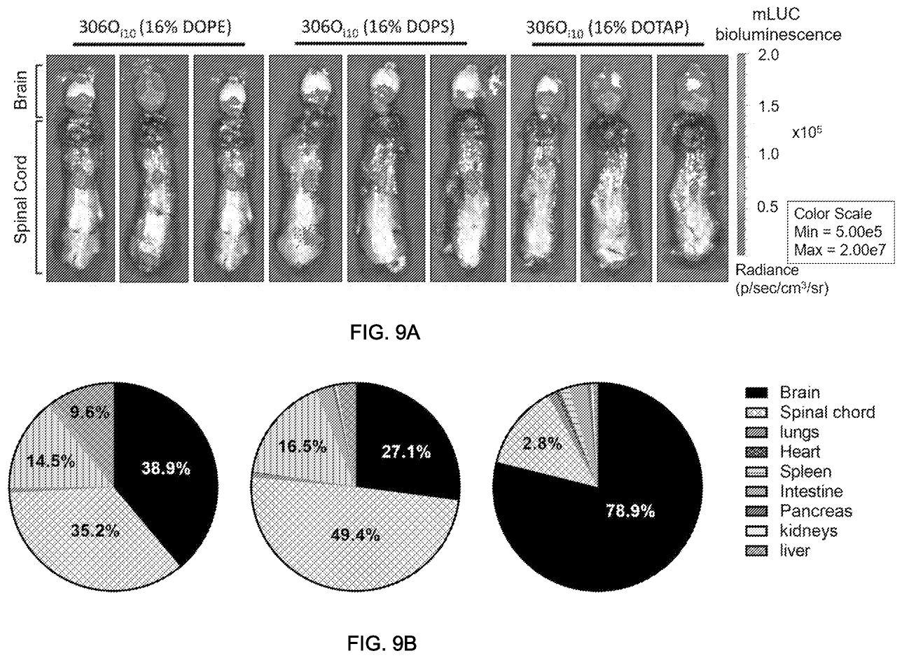

As shown in FIGS. 9A-9B, all LNPs trafficked to the brain regardless of the helper lipid charge, with DOTAP producing the most brain luciferase expression.

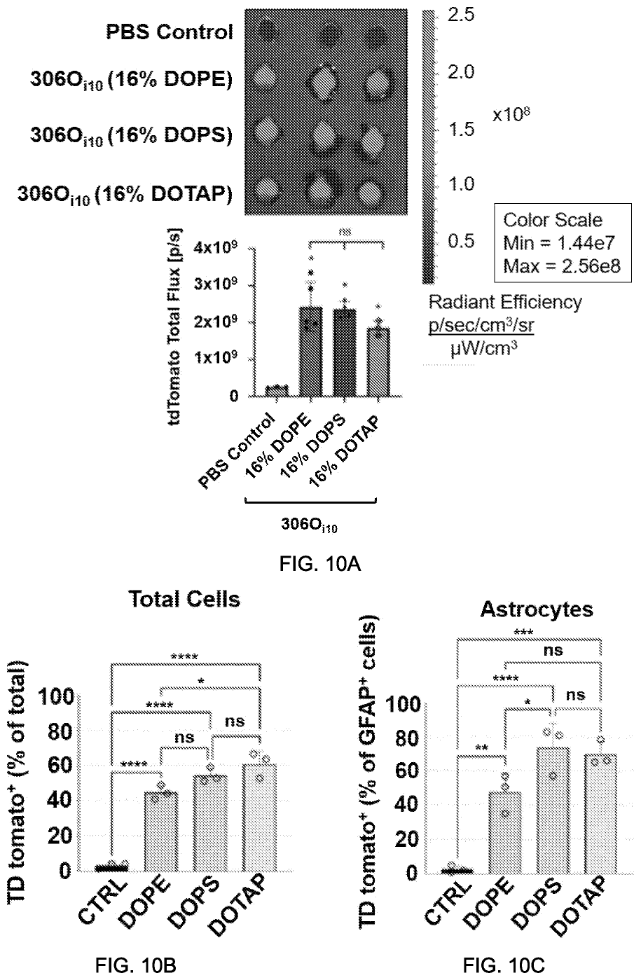

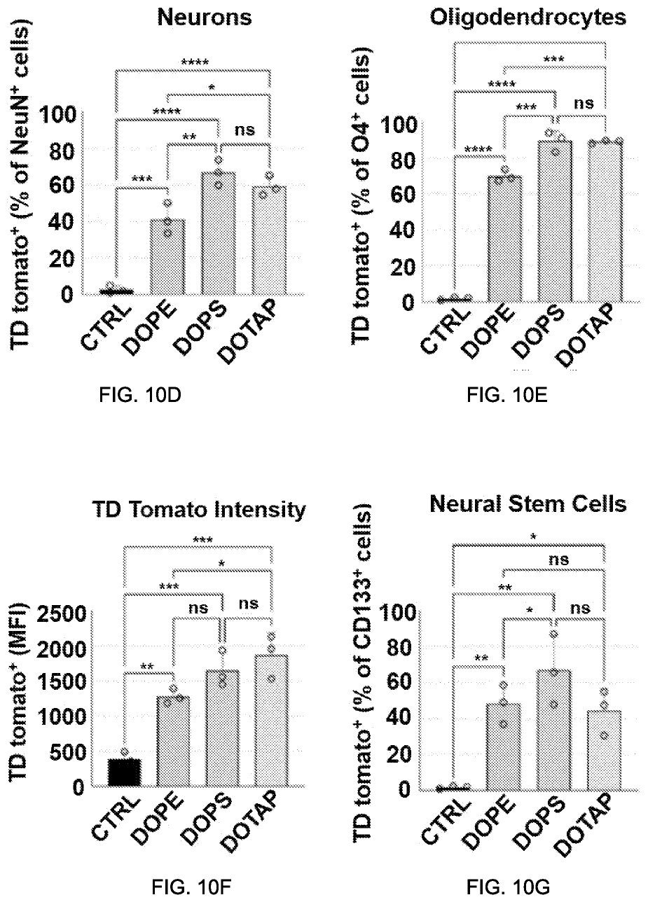

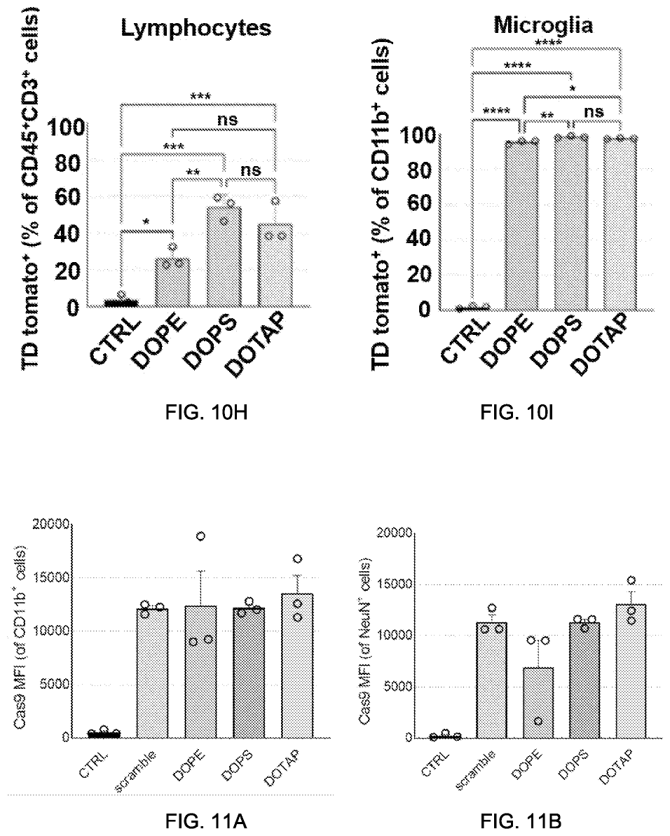

The LNPs with mCre administered to Ai9 mice expressed tdTomato upon Cre-mediated recombination. This revealed which cells translate mCre, not the amount of protein expressed (FIGS. 10A-10I). 85-95% of microglia (CD11b+), 35-75% of astrocytes (GFAP+), 35-75% Neurons (NeuN+), 35-85% of Neural stem cells (CD133+), 60-95% of oligodendrocytes, and 15-60% of lymphocytes (CD45+CD3+) translated mCre. Thus, intrathecal injection of mRNAs in LNPs successfully transfected brain cells in vivo.

### Cas9-Mediated Gene Knockout in Mice

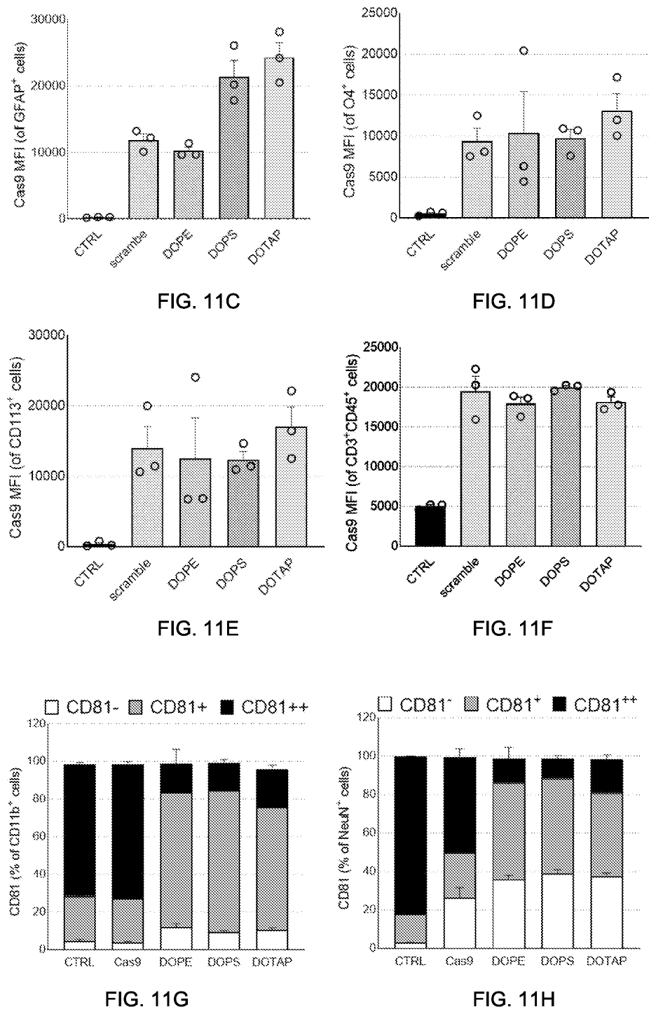

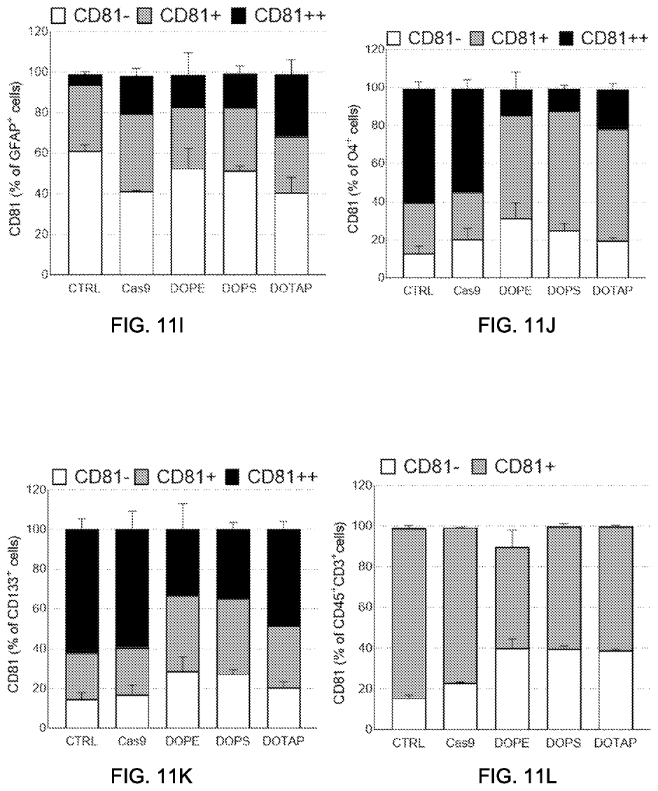

As shown in FIGS. 11A-11L, CD81 was successfully knocked out in microglial cells, neurons and oligodendrocytes.

### Example 4—In Vivo Intrathecal LNP Administration

### Materials and Methods

LNPs were independently formulated with (1) 16 mol % Ceramide, 35 mol % ionizable lipidoid (306_{Oi10} as a proof-of concept example), 46.5 mol % cholesterol, and 2.5 mol % C₁₄-PEG₂₀₀₀; (2) 16 mol % glucosyl ceramide, 35 mol % ionizable lipidoid (306_{Oi10} as a proof-of concept example), 46.5 mol % cholesterol and 2.5 mol % C₁₄-PEG₂₀₀₀; (3) 16 mol % galactosyl ceramide, 35 mol % ionizable lipidoid (306_{Oi10} as a proof-of concept example), 46.5 mol % cholesterol and 2.5 mol % C₁₄-PEG₂₀₀₀; or (4) 16 mol % lactosyl ceramide, 35 mol % ionizable lipidoid (306_{Oi10} as a proof-of concept example), 46.5 mol % cholesterol and 2.5 mol % C₁₄-PEG₂₀₀₀. The mRNA was mLuc, mCre, a combination of mLuc and mCherry, or a combination of mRNA for Cas9 and sgRNA for murine CD81. The LNPs were prepared according to the method described in Example 1. The LNPs were formulated to contain 0.5 mg/kg mRNA. The LNPs containing a combination of mRNA for Cas9 and sgRNA for murine CD81 were formulated to contain 0.5 mg/kg mRNA for Cas9 and 0.05 mg/kg sgRNA for murine CD81, where ratio of cas9 mRNA to sgRNA was 4.7:1 (w/w).

Evaluation of mRNA Delivery to the Brain

Male C57BL/6 (Charles River) mice or male and female Ai9 reporter mice (Jackson Laboratory) of at least 6 weeks of age were used in vivo experiments. C57BL/6NCrl mice were independently injected intrathecally with (1) Ceramide helper lipid LNPs containing mLuc; (2) glucosyl ceramide helper lipid LNPs containing mLuc; (3) galactosyl ceramide helper lipid LNPs containing mLuc; (4) lactosyl ceramide helper lipid LNPs containing mLuc; (5) Ceramide helper lipid LNPs containing a combination of mLuc and mCherry; (6) glucosyl ceramide helper lipid LNPs containing a combination of mLuc and mCherry; (7) galactosyl ceramide helper lipid LNPs containing a combination of mLuc and mCherry; or (8) lactosyl ceramide helper lipid LNPs containing a combination of mLuc and mCherry. Male or female Ai9 mice were independently injected intrathecally with (1) Ceramide helper lipid LNPs containing mCre; (2) glucosyl ceramide helper lipid LNPs containing mCre; (3) galactosyl ceramide helper lipid LNPs containing mCre; or (4) lactosyl ceramide helper lipid LNPs containing mCre. After 1 hour, 3 hours, 6 hours, 12 hours, 18 hours, 24 hours, or 48 hours post-injection, the mice injected with LNPs containing mLuc were injected intraperitoneally with 130 μL of 30 mg/mL D-luciferin. After 6 hours post-injection, the mice injected with LNPs containing mLuc and mCherry were injected intraperitoneally with 130 μL of 30 mg/mL D-luciferin. After 96 hours post-injection, the mice injected with LNPs containing mCre were injected intraperitoneally with 130 μL of 30 mg/ml D-luciferin. The mice were euthanized and the organs were prepared and analyzed as described in Example 2. Single cells suspensions were collected from the brains of the mice treated with the LNPs containing mCre and were prepared for flow cytometry analysis. Brains were also fixed, embedded, and stained for central nervous system cells and imaged using confocal microscopy for tdTomato fluorescence.

### Cas9-Mediated Gene Knockout in Mice

Male C57BL/6NCrl mice were independently injected intrathecally with (1) Ceramide helper lipid LNPs containing mRNA for Cas9 and sgRNA for murine CD81; (2) galactosyl ceramide helper lipid LNPs containing mRNA for Cas9 and sgRNA for murine CD81; or (3) lactosyl ceramide helper lipid LNPs containing mRNA for Cas9 and sgRNA for murine CD81. After one week post-injection, the C57BL/6NCrl mice were injected intraperitoneally with 130 μL of 30 mg/mL D-luciferin. The mice were euthanized and the organs were prepared and analyzed as described in Example 2. The brains from the mice were harvested, mechanically digested into single cell suspensions and analyzed using flow cytometry to assess for CD81 knockout. To assess successful expression of Cas9, a separate set of animals were euthanized at 24 hours post LNP administration.

### Toxicity Assessment

Male and female C57BL/6NCrl mice were independently injected intrathecally with (1) Ceramide helper lipid LNPs containing mLuc; (2) glucosyl ceramide helper lipid LNPs containing mLuc; (3) galactosyl ceramide helper lipid LNPs containing mLuc; (4) lactosyl ceramide helper lipid LNPs containing MLuc; or (5) PBS. Blood was drawn from the mice 1 hour, 3 hours, 6 hours, 24 hours, or 48 hours post-injection. The serum was separated and assessed for inflammatory cytokines using an enzyme-linked immunosorbent assay (ELISA).

### Results and Discussion

mRNA Delivery to the Brain

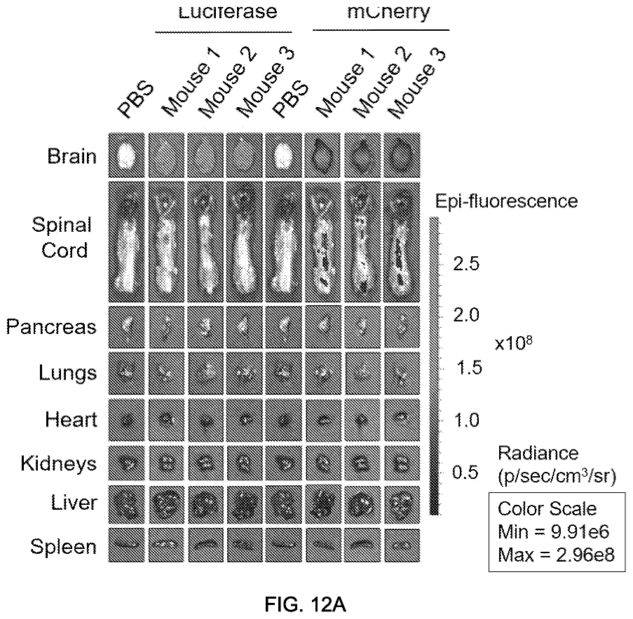

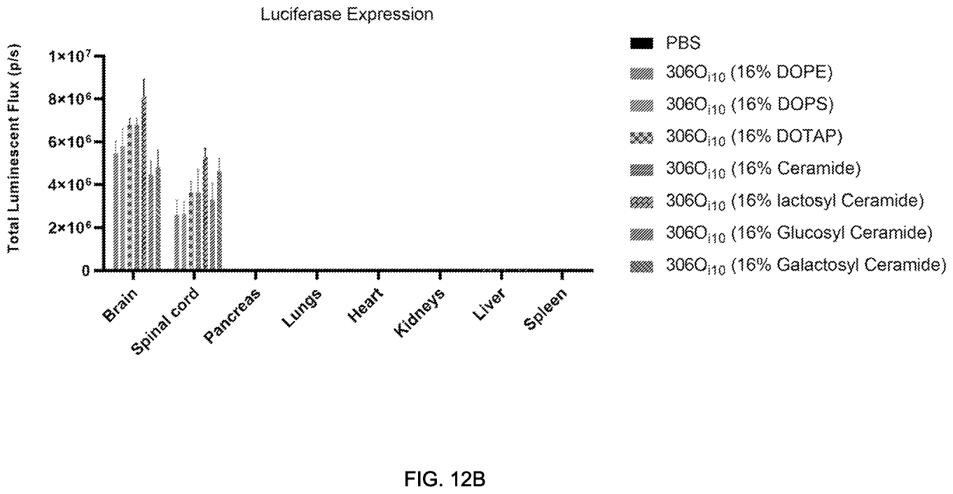

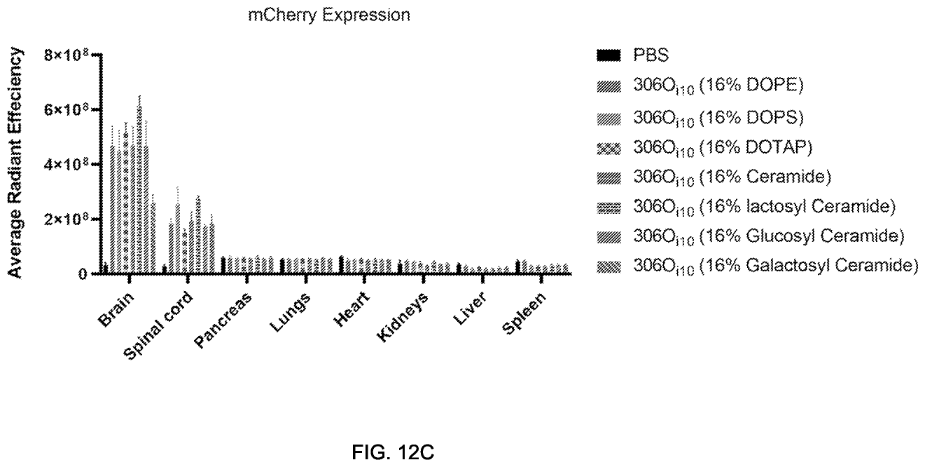

Multiplex mRNA delivery of the LNPs containing mLuc and mCherry was analyzed at 6 hours post injection. The resulting data is shown in FIGS. 12A-12B, which shows successful transfection with mRNAs for luciferase and mCherry. The data from the LNPs prepared with Ceramide, glucosyl ceramide, lactosyl ceramide, and galactosyl ceramide was compared to LNPs prepared with DOPE, DOPS, and DOPAP. Thus, multiple mRNAs were delivered to the brain via intrathecal injection.

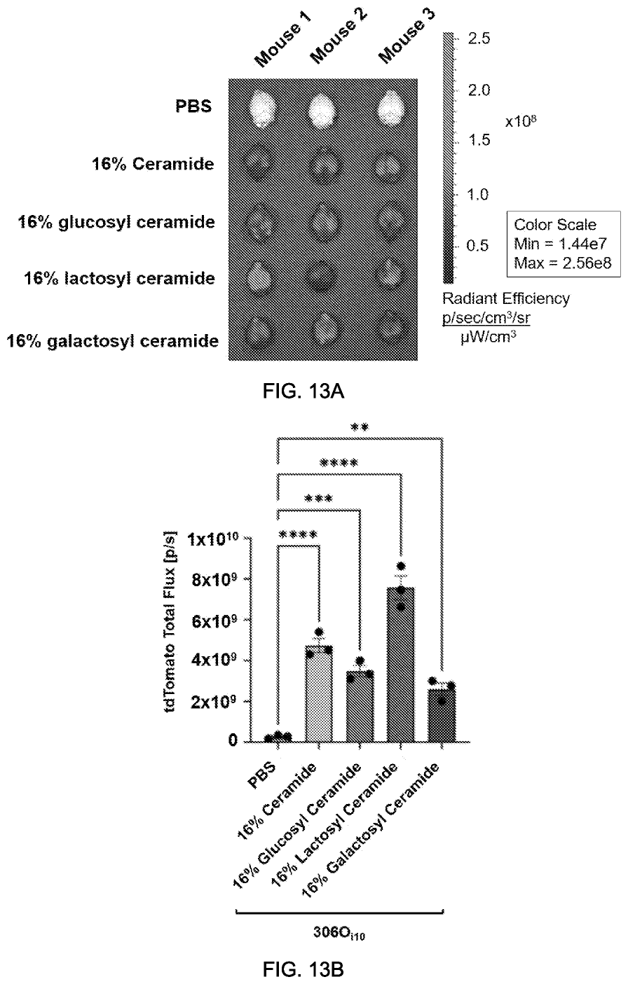

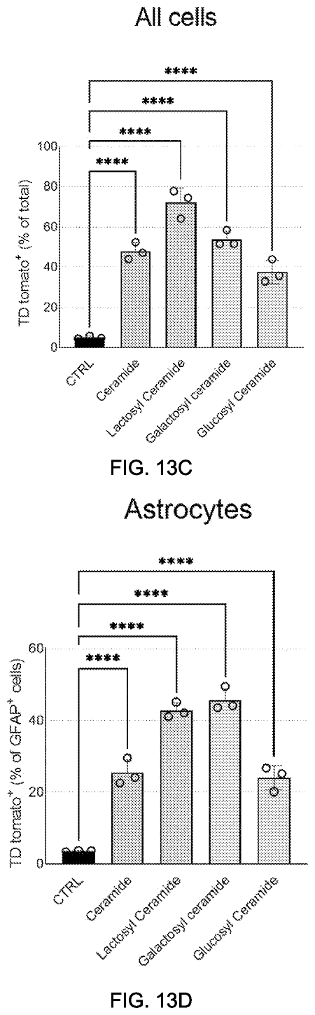

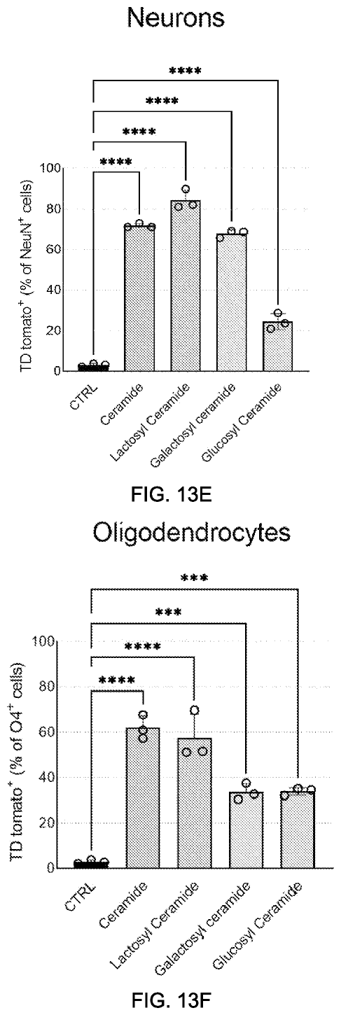

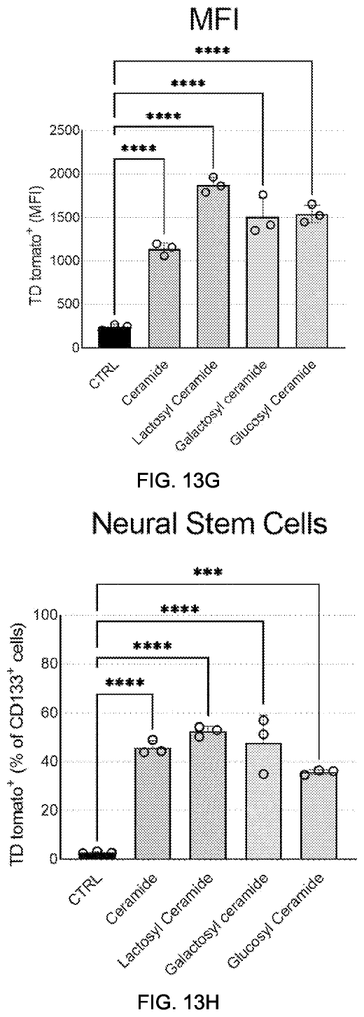

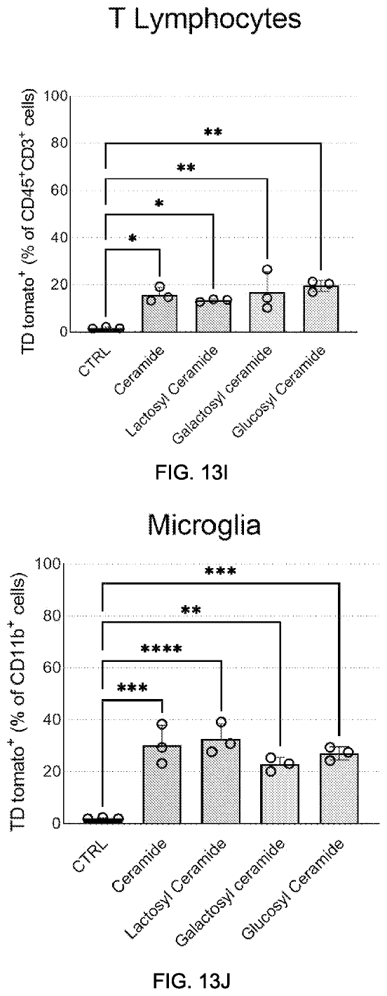

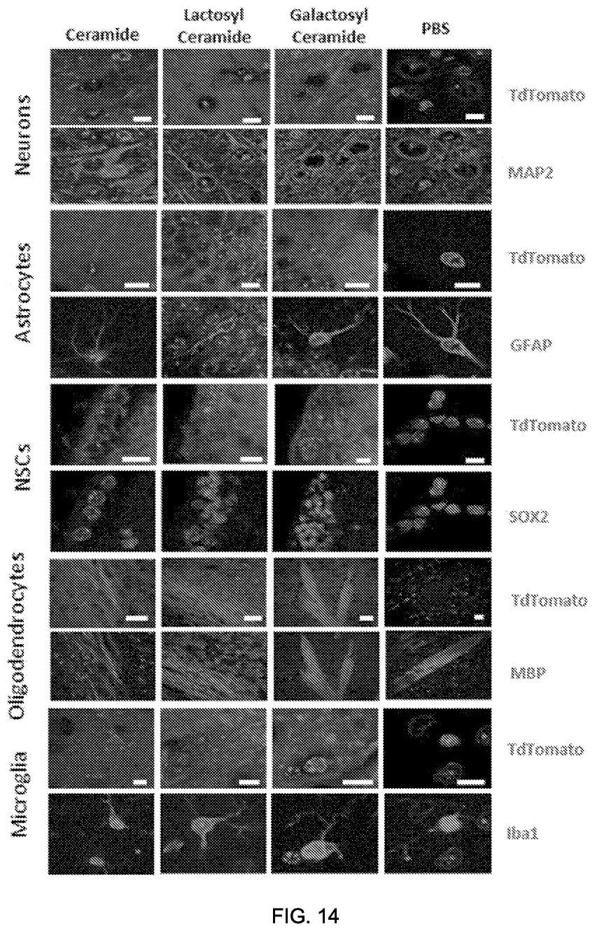

The lipid LNPs formulated with Ceramide, glucosyl ceramide, galactosyl ceramide, or lactosyl ceramide all successfully transfected mouse brains with mCre after a single intrathecal injection (FIGS. 13A-13J). The LNPs formulated with Ceramide and lactosyl ceramide had the highest transfection efficacy among the LNPs tested. All of the LNPs were able to transfect around 80% of neurons in the brain, as confirmed using immunostaining for different central nervous system cells (CNS) (see FIG. 14). Overall, the data indicated that intrathecal injection of mRNAs in LNPs successfully transfects neurons and other brain cells in vivo.

### Cas9-Mediated Gene Knockout in Mice

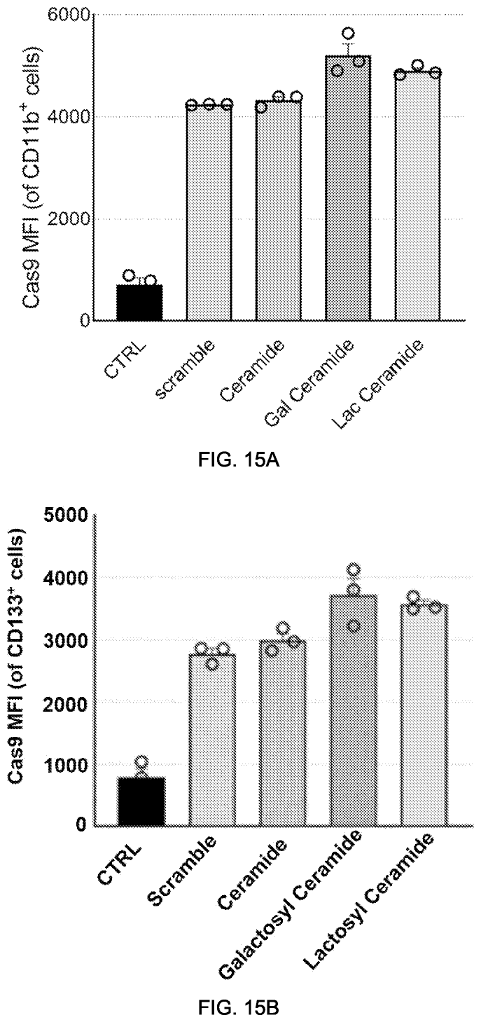

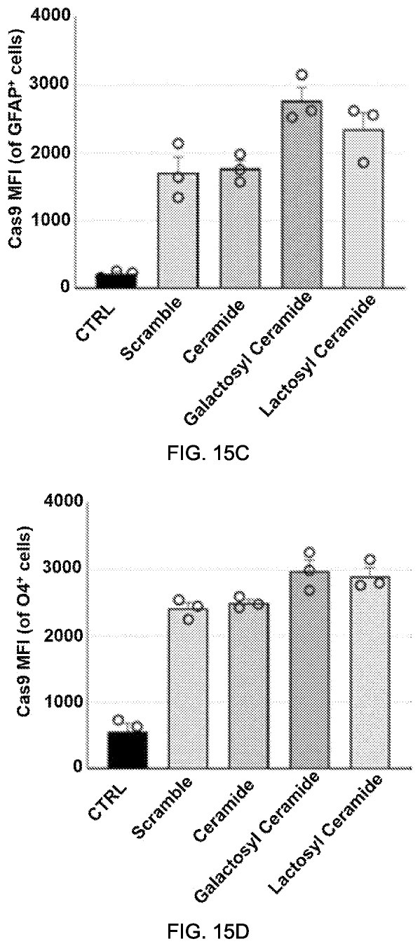

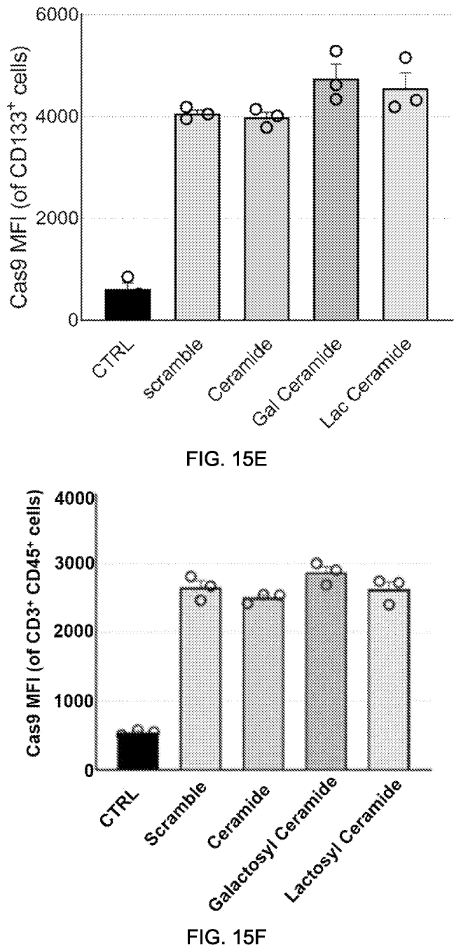

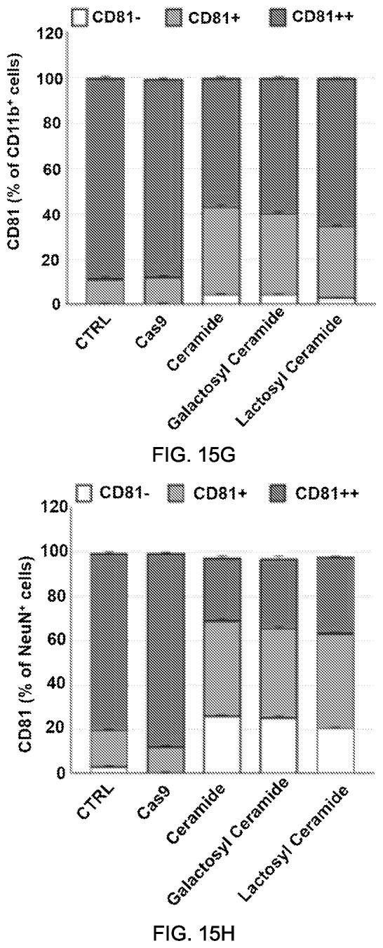

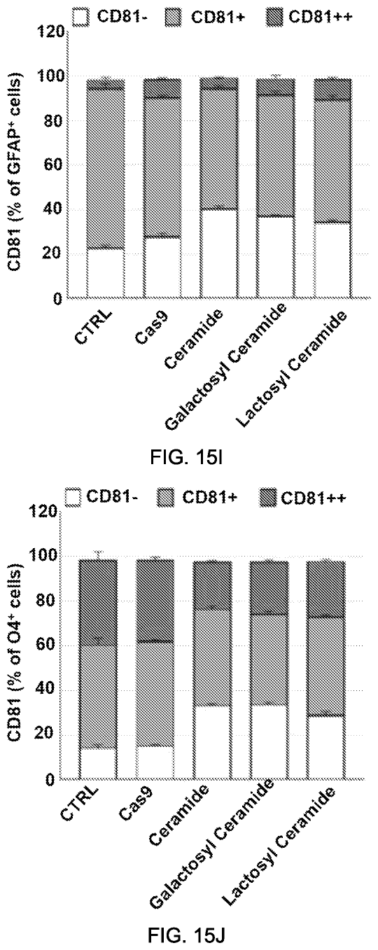

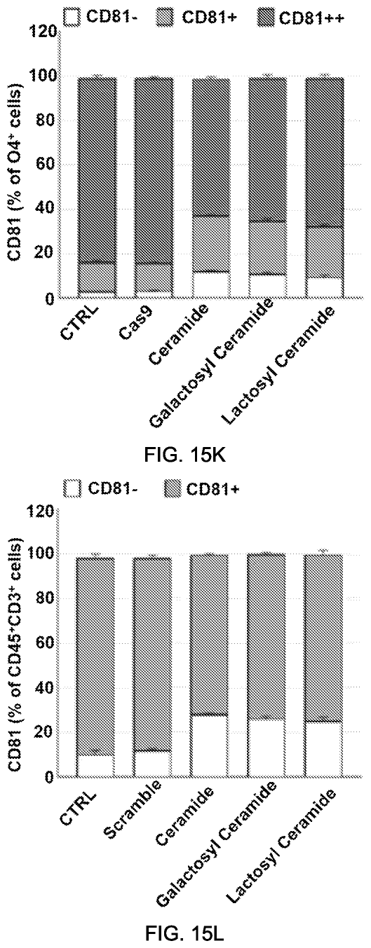

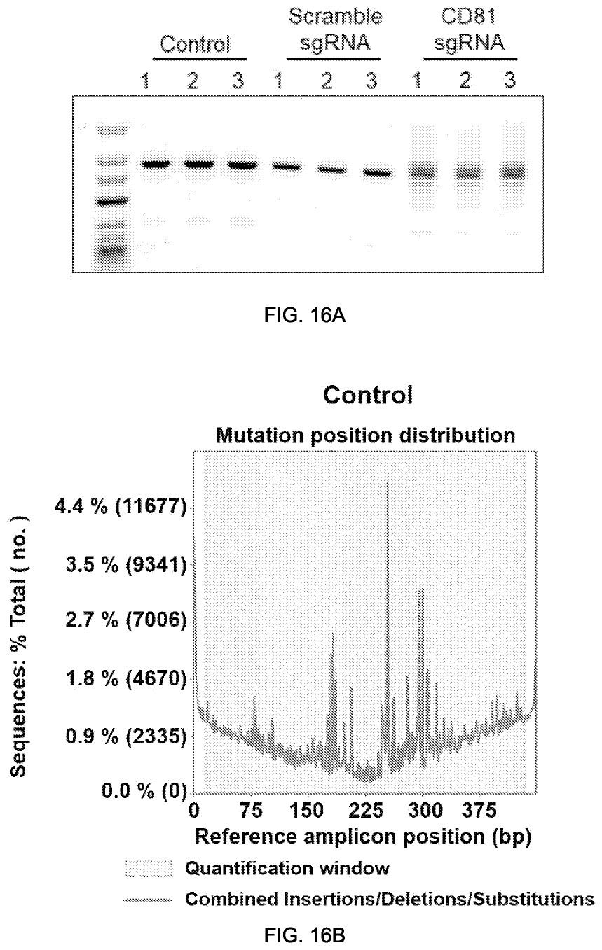

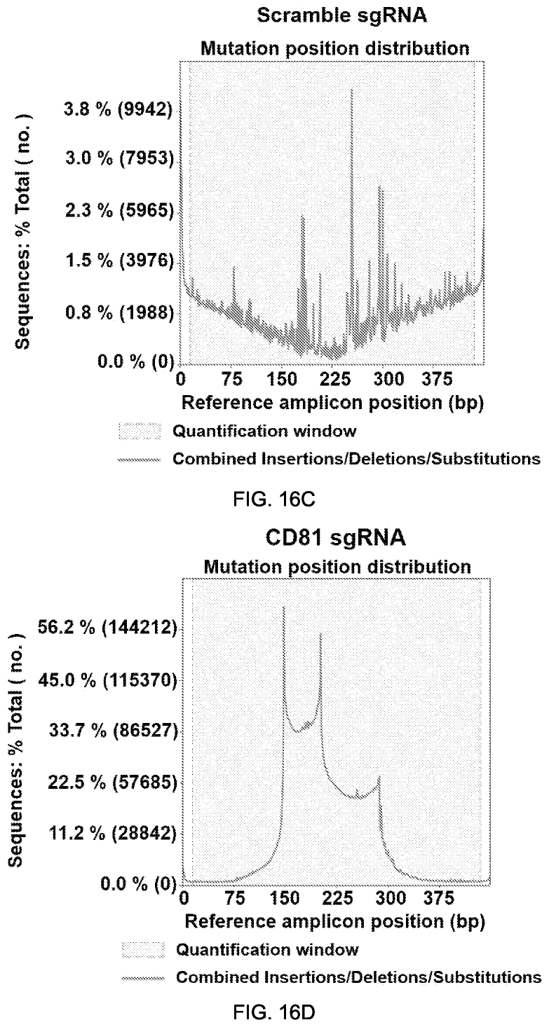

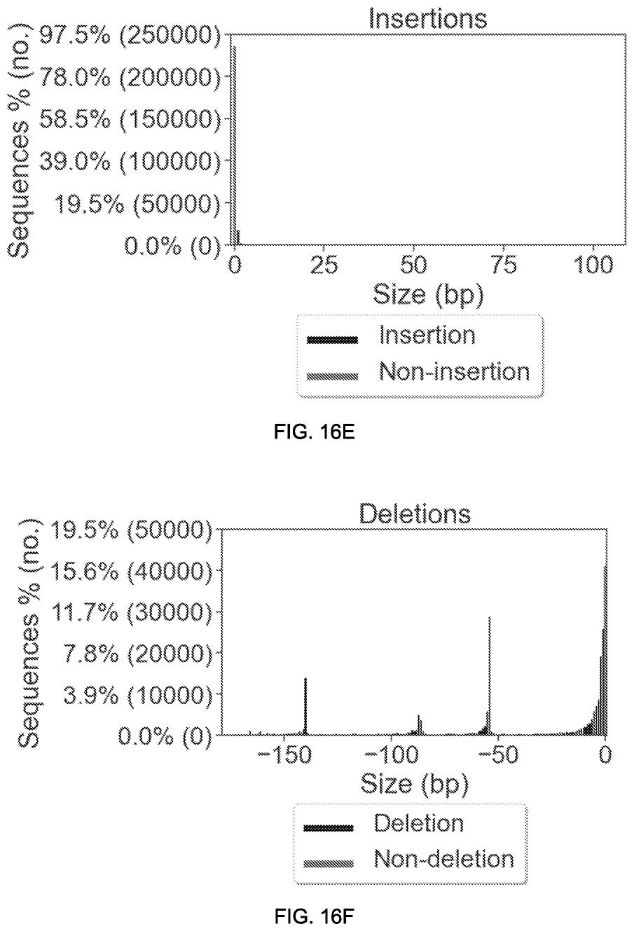

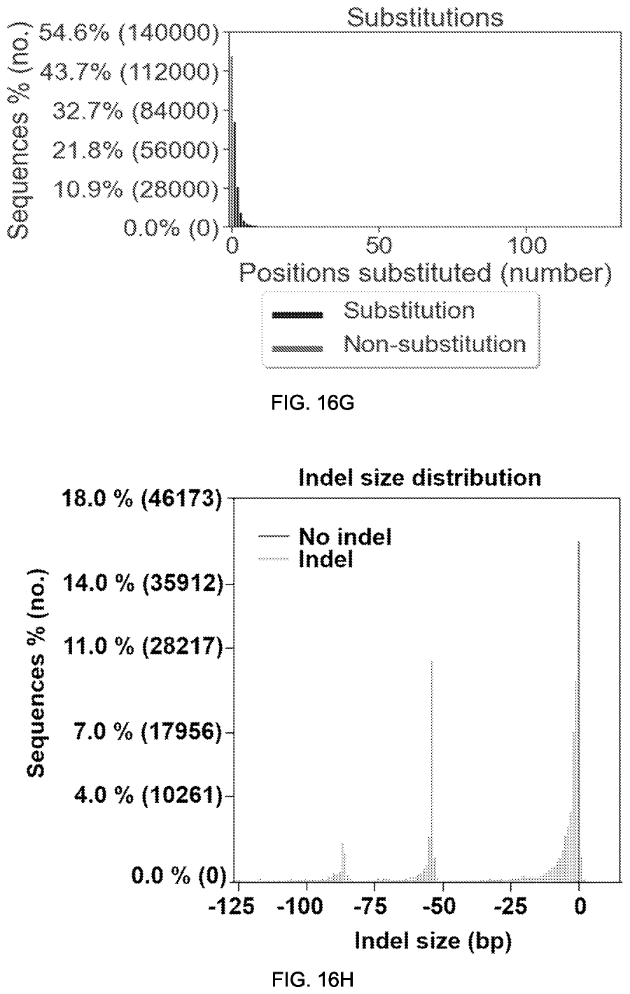

As shown in FIGS. 15A-15L, most of the CD81 gene editing happened in neurons, as compared to other cell types. In addition, the CD81 gene editing in the mouse brain was confirmed by gene sequencing, as provided in FIGS. 16A-16H. The multiple bands in the CD81 sgRNA LNPs of FIG. 16A is indicative of Cas9 mediated gene cuts. There were indels in the CD81 gene locus corresponding to Cas9-mediated mediated editing.

### Toxicity Assessment

The data showed that although there was a 1-2 fold increase in some inflammatory cytokines within 3-6 hours of LNP injection, this was resolved within 24 hours of injection and did not result in any severe inflammatory reaction. However, the transient increase in the inflammatory cytokines was also observed in the PBS control group suggesting that the administration route caused an inflammatory reaction rather than the LNP formulations.

Thus, the intrathecal administration of LNPs formulated with Ceramide, glucosyl ceramide, galactosyl ceramide, or lactosyl ceramide was well tolerated by mice.

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments, but rather by the appended claims as originally filed.

1. A method of delivery of a therapeutic agent to tissue of the central nervous system (CNS) of a patient, comprising administering to tissue of the patient's CNS a composition comprising a lipid-containing particle, comprising a therapeutic agent, the lipid-containing particle further comprising:
a sphingolipid helper lipid;
cholesterol or a derivative thereof;
a PEG-based compound; and
an ionizable lipidoid.

2. (canceled)

3. The method of claim 1, wherein the lipid-containing particle is administered intrathecally, intracerebrally, or to a patient's brainstem.

4. The method of claim 1, wherein the sphingolipid is a glucosyl ceramide, a galactosyl ceramide, a lactosyl ceramide, a sphingomyelin, a ceramide phosphoethanolamine, a sphingosyl-phosphorylcholine, a sphingosine, a glycosphingolipid, or any combination of two or more of the preceding.

5-7. (canceled)

8. The method of claim 1, wherein the ionizable lipidoid is one or more of 306_{Oi10}; 306O₁₀; 503Oᵢ₁₀; 402O_{6,10}; 500X₁; 500Oᵢ₁₀; 306O₁₁; 306Oi10; 306O₁₂; 200X₆; 516Oᵢ₁₀; 500O_{1,1,8}; 514X₆; 306O₁₄; 501X₁; 205O₁₆; 500O₁₃; 113Oᵢ₁₀; 306O₁₆; 306O₁₃; 205O₁₈; 509X₇; 501Oᵢ₁₀; 503Oᵢ₁₀; 500O₁₄; 113Oᵢ₁₀; 509X₁; 509X₃; 501X₂; 402O_{6,10}; 516O_{4,8}; 402X₈; 501O_{1,1,8}; and 509O_{1,1,8}.

9. (canceled)

10. The method of claim 1, wherein the ionizable lipidoid is 306_{Oi10}.

11. (canceled)

12. The method of claim 1, wherein the therapeutic agent is a nucleic acid.

13-14. (canceled)

15. The method of claim 12, wherein the nucleic acid comprises DNA, an RNAi reagent, a dsRNA, an siRNA, an shRNA, a miRNA, an antisense RNA, a guide RNA (gRNA), a long non-coding RNAs (lncRNA), a base editing gRNA (beRNA), a prime editing gRNA (pegRNA), a messenger RNA (mRNA) encoding Cas9 or a Cas9 fusion protein for base editing or prime editing, or a transfer RNA (tRNA).

16. (canceled)

17. The method of claim 12, wherein the nucleic acid encodes a heme oxygenase-1 (HO1), brain-derived neurotrophic factor (BDNF), or tumor necrosis factor-related apoptosis-inducing ligand (TRAIL) protein.

18. (canceled)

19. The method of claim 1, wherein the lipid-containing particle is a lipid nanoparticle, comprising, by mol % of the sphingolipid helper lipid, the cholesterol or a derivative thereof, the PEG-based compound, and the lipidoid:
from 5 to 95 mol % of the sphingolipid helper lipid;
from 5 to 75 mol % of the cholesterol or a derivative thereof;
from 0.1 to 50 mol % of the PEG-based compound; and
from 5 to 90 mol % of the ionizable lipidoid.

20. (canceled)

21. The method of claim 1, wherein the PEG-based compound is one or more of: a PEGylated fatty acid, PEG-ceramide, PEG-DMG, PEG-PE, poloxamer, DSPE carboxy PEG C14 PEG2000 DMG, C15 PEG2000 DMG, C16 PEG2000 DMG, C18 PEG2000 DMG, C14 PEG 2000 ceramide, C15 PEG2000 ceramide, C16 PEG2000 ceramide, C18 PEG2000 ceramide, C14 PEG2000 PE, C15 PEG2000 PE, C16 PEG2000 PE, C18 PEG2000 PE, C14 PEG350 PE, C14 PEG5000 PE, poloxamer F-127, poloxamer F-68, poloxamer L-64, and DSPE carboxy PEG.

22-24. (canceled)

25. A lipid-containing particle, comprising a therapeutic agent, the lipid-containing particle further comprising:
a sphingolipid helper lipid;
cholesterol or a derivative thereof;
a PEG-based compound; and
an ionizable lipidoid.

26. (canceled)

27. The lipid-containing particle of claim 25, wherein the sphingolipid is a CNS sphingolipid, a glucosyl ceramide, a galactosyl ceramide, a lactosyl ceramide, a sphingomyelin, a ceramide phosphoethanolamine, a sphingosyl-phosphorylcholine, a sphingosine, a glycosphingolipid, or any combination of two or more of the preceding.

28-30. (canceled)

31. The lipid-containing particle of claim 25, wherein the ionizable lipidoid is one or more of 306_{Oi10}; 306O₁₀; 503Oᵢ₁₀; 402O_{6,10}; 500X₁; 500Oᵢ₁₀; 306O₁₁; 306Oi10; 306O₁₂; 200X₆; 516Oᵢ₁₀; 500O_{1,1,8}; 514X₆; 306O₁₄; 501X₁; 205O₁₆; 500O₁₃; 113Oᵢ₁₀; 306O₁₆; 306O₁₃; 205O₁₈; 509X₇; 501Oᵢ₁₀; 503Oᵢ₁₀; 500O₁₄; 113Oᵢ₁₀; 509X₁; 509X₃; 501X₂; 402O_{6,10}; 516O_{4,8}; 402X₈; 501O_{1,1,8}; and 509O_{1,1,8}.

32. (canceled)

33. The lipid-containing particle of claim 25, wherein the ionizable lipidoid is 306_{Oi10}.

34. (canceled)

35. The lipid-containing particle of claim 25, wherein the therapeutic agent is a nucleic acid.

36-37. (canceled)

38. The lipid-containing particle of claim 35, wherein the nucleic acid comprises DNA, an RNAi reagent, a dsRNA, an siRNA, an shRNA, a miRNA, an antisense RNA, a guide RNA (gRNA), a long non-coding RNAs (lncRNA), a base editing gRNA (beRNA), a prime editing gRNA (pegRNA), a messenger RNA (mRNA) encoding Cas9 or a Cas9 fusion protein for base editing or prime editing, or a transfer RNA (tRNA).

39. (canceled)

40. The lipid-containing particle of claim 35, wherein the nucleic acid encodes a heme oxygenase-1 (HO1), brain-derived neurotrophic factor (BDNF), or tumor necrosis factor-related apoptosis-inducing ligand (TRAIL) protein.

41. (canceled)

42. The lipid-containing particle of claim 25, wherein the lipid-containing particle is a lipid nanoparticles, comprising, by mol % of the sphingolipid helper lipid, the cholesterol or a derivative thereof, the PEG-based compound, and the lipidoid:
from 5 to 95 mol % of the sphingolipid helper lipid;
from 5 to 75 mol % of the cholesterol or a derivative thereof;
from 0.1 to 50 mol % of the PEG-based compound; and
from 5 to 90 mol % of the ionizable lipidoid.

43. (canceled)

44. The lipid-containing particle of claim 25, wherein the PEG-based compound is one or more of: a PEGylated fatty acid, PEG-ceramide, PEG-DMG, PEG-PE, poloxamer, or DSPE carboxy PEG, C14 PEG2000 DMG, C15 PEG2000 DMG, C16 PEG2000 DMG, C18 PEG2000 DMG, C14 PEG 2000 ceramide, C15 PEG2000 ceramide, C16 PEG2000 ceramide, C18 PEG2000 ceramide, C14 PEG2000 PE, C15 PEG2000 PE, C16 PEG2000 PE, C18 PEG2000 PE, C14 PEG350 PE, C14 PEG5000 PE, poloxamer F-127, poloxamer F-68, poloxamer L-64, and DSPE carboxy PEG.

45-48. (canceled)

49. A method of treating a patient having ischemia, stroke, ischemia/reperfusion injury, neuroinflammation, a neurodegenerative disease, a monogenic neurological disorder, or a cancer, comprising administering to the patient an effective amount of the lipid nanoparticle as claimed in claim 25, thereby treating the ischemia, stroke, ischemia/reperfusion injury, neuroinflammation, neurodegenerative disease, monogenic neurological disorder, or a cancer in the patient.
